(19) United States
(12) Patent Application Publication
(10) Pub. No.: US 2024/0174642 A1
(43) Pub. Date: May 30, 2024

(54) HETEROCYCLIC COMPOUNDS CAPABLE OF ACTIVATING STING

(71) Applicant: Boehringer Ingelheim International GmbH, Ingelheim am Rhein (DE)

(72) Inventors: Keith Andrew Newton GRAHAM, Berlin (DE); Sebastian CAROTTA, Baden (AT); Georg DAHMANN, Biberach an der Riss (DE); Cédrickx GODBOUT, Schemmerhofen (DE); Sandra Ruth HANDSCHUH, Biberach an der Riss (DE); Herbert NAR, Ochsenhausen (DE); Thorsten OOST, Biberach an der Riss (DE); Ulrich REISER, Vienna (AT); Esther SCHMIDT, Burgrieden (DE); Matthias TREU, Vienna (AT)

(21) Appl. No.: 18/493,142

(22) Filed: Oct. 24, 2023

(30) Foreign Application Priority Data
Oct. 26, 2022 (EP) 22203741.8

(51) Int. Cl. C07D 401/14; A61P 35/00; C07D 413/14; C07D 471/08; C07D 471/18

(52) U.S. Cl. CPC C07D 401/14; A61P 35/00; C07D 413/14; C07D 471/08; C07D 471/18

(57) ABSTRACT

The present invention relates to compounds of formula (I) which are capable of activating STING (Stimulator of Interferon Genes).

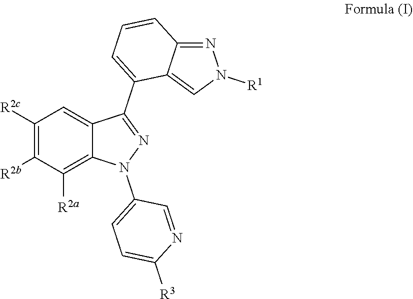

The present invention further relates to pharmaceutical compositions comprising at least a compound of formula (I), as well as the use of these compounds or the pharmaceutical compositions as a medicament, e.g., for treating canine or feline cancer, or as vaccine adjuvants.

### SEQUENCE LISTING

The instant application contains a Sequence Listing which has been submitted electronically in XML format and is hereby incorporated by reference in its entirety. Said XML copy, created on Sep. 12, 2023, is named “23-0024-US-1-2023-10-24-Sequence-Listing.XML” and is 2,139 bytes in size.

### FIELD OF THE INVENTION

The present invention relates to small molecules capable of activating STING (Stimulator of Interferon Genes), and their salts. Specifically, the present invention relates to heterocyclic compounds capable of activating STING. Furthermore, the invention relates to pharmaceutical compositions and combinations comprising these compounds, as well as their use as a medicament. These compounds and pharmaceutical compositions comprising at least one of these compounds may be suitable as a medicament, e.g., for the therapy of cancer such as canine and/or feline cancer, and as vaccine adjuvant, e.g., for use in swine. Therefore, the invention also relates to compounds and pharmaceutical compositions comprising at least one of these compounds for use in treating feline or canine cancer.

### BACKGROUND OF THE INVENTION

STING is one of the pattern-recognition receptors (PRRP) which plays a central role in the innate immune system, distinguishing pathogens and host cells by detecting extracellular and intracellular danger signals including damage-associated molecular patterns (DAMP) and pathogen-associated molecular patterns (PAMP). These recognition processes constitute the first line of defense against viral and bacterial infections and malignant cells. However, pathogens, as well as cancer cells, have evolved ways to evade recognition by the immune system. The aim of immunotherapies is thus to initiate an antigen specific immune response or to re-activate a pre-existing response in certain cell types of the immune system against the pathogenic invaders or cancerous cells.

Among the PRRPs, STING (also known as TMEM173, MPYS, MITA, ERIS) belongs to the family of nucleic acid sensors and is the adaptor for cytosolic DNA signaling. In mammalian cells, in a healthy state, DNA is compartmentalized in the nucleus. In pathogenic situations, such as invasions of DNA-containing pathogens, or in malignant cells, DNA is present in the cytoplasm. Here, STING is critical for detecting the above-described cytosolic DNA and to induce an immune reaction against the pathogenic event.

Being an innate immune defense mechanism member, STING is expressed in almost all cell types especially endothelial, epithelial and immune cells such as macrophages and dendritic cells. A predominant expression of STING in dogs and cats is observed in spleen, lung, blood, lymph nodes and brain (Zhang et al, Microbial Pathogenesis, 113, 202-208, 2017; Zhang et al, Veterinary Immunology and Immunopathology 169, 54-62, 2016).

In its basal state, STING exists as a dimer with its N-terminal domain anchored in the ER and the C-terminal domain residing in the cytosol. Cyclic dinucleotides (CDNs), generated by the protein cyclic GMP-AMP Synthase (cGAS) are the natural ligands of STING (Ablasser et al, Nature 498, 380-384, 2013). Binding of CDNs to STING induces conformational changes which allows the binding and activation of the TANK binding kinase (TBK1) and interferon regulatory factor 3 (IRF3), followed by the relocalization from the ER to perinuclear endosomes (Liu et al, Science 347, Issue 6227, 2630-1-2630-14, 2015). Phosphorylation of the transcription factor IRF3 and NF-kB byTBK1 results in expression of multiple cytokines, including type I interferon (IFN).

Type I IFN production by antigen presenting cells, and other cell types, is considered a key event in the activation of T cells and thereby the differentiation of antigen specific effector CD4 and CD8 T cells. It was shown that the lack of type I IFN resulted in a reduced T cell dependent immune response against viral infections or tumor cells (Zitvogel et al, Nature Reviews Immunology 15, 405-414, 2015). On the other hand, the presence of a type I IFN signature during cancer therapy is associated with increased numbers of tumor infiltrating T cells and potentially favorable clinical outcome (Sistigu et al, Nature Medicine 20, 1301-1309, 2014).

The anti-tumor impact of type I interferons has been investigated in vitro and in vivo for feline and canine cancers. Based on field studies where human recombinant interferons were administrated to tumor bearing dogs (lymphoma, fibrosarcoma, osteosarcoma, mycosarcoma, liposarcoma), delayed or prevented local relapses and metastases were documented (Klotz et al, Veterinary Immunology and Immunopathology 191, 80-93, 2017).

Efficient secretion of type I IFN in the tumor microenvironment and the induction of a T cell dependent immune response against cancer cells depends on the presence of STING, as shown in recent studies in mice (Woo et al, Immunity 41, 5, 830-842, 2014; Corrales et al, Cell Reports 11, 1018-1030, 2015; Deng et al, Immunity 41, 5, 843-852, 2014). The deletion of STING resulted in reduced type I IFN levels in the tumor microenvironment and in a reduced anti-tumor effect in several mouse tumor models, thereby highlighting the importance of the presence of type I IFN. On the other hand, the specific activation of STING resulted in an improved, antigen specific T cell immune response against cancer cells.

Type I interferons can significantly enhance anti-tumor immune responses by inducing activation of both the adaptive and the innate immune cells.

Given the importance of type I IFN in several malignancies including viral infections and cancer therapy, strategies that allow the specific activation of STING are of therapeutic interest. STING activation may be synergistic with various approved chemotherapeutic agents or other anti-cancer therapies such as radiotherapy (Wu et al., Med Res Rev 2020 May; 40(3):1117-1141) or with infectious disease therapies.

In the prior art, small molecule modulators of STING are for example described in WO 2020/075790.

### SUMMARY OF THE INVENTION

Compounds according to the present invention are novel activators of STING as demonstrated in an ex vivo system using canine whole blood.

In one aspect, the present invention relates to a compound of formula (I)

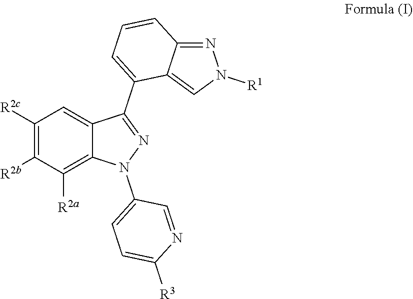

wherein
R¹ is a —C₁₋₆-alkyl or —C₃₋₆ cycloalkyl;
R^{2a} is selected from among the group consisting of —H or —C₁₋₆-alkyl;
R^{2b} is selected from among the group consisting of —H, —C₁₋₆-alkyl, —C₁₋₆-alkylene-OH, —C(O)OH, —C(O)O—C₁₋₆-alkyl and -pyrazolyl-C₁₋₆-alkyl;
R^{2c} is —H or —C₁₋₆-alkyl;
R³ is selected from among the group consisting of (* indicates the point of attachment):

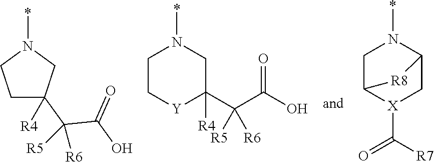

R⁴ is selected from H, —C₁₋₆-alkyl, and C₃₋₆ cycloalkyl;
R⁵ is selected from H, —C₁₋₆-alkyl, and C₃₋₆ cycloalkyl;
is R⁶ is selected from H, —C₁₋₆-alkyl, and C₃₋₆ cycloalkyl;
R⁷ is selected from H, —C₁₋₆-alkyl, C₃₋₆ cycloalkyl and —OH;
R⁸ is (CH₂)ₙ, wherein n is an integer of 1-3, preferably 1 or 2;
X is CH or N; and
Y is —O—, —S—, —S(O)—, —S(O)₂—;
or a pharmaceutically acceptable salt thereof.

In another aspect, the invention relates to a pharmaceutical composition comprising at least one compound according to formula (I) or a pharmaceutically acceptable salt thereof and a pharmaceutically acceptable carrier.

In another aspect, the invention relates to a compound according to formula (I) or a pharmaceutically acceptable salt thereof or a pharmaceutical composition including the same for use as a medicament.

In another aspect, the invention relates to a compound according to formula (I) or a pharmaceutically acceptable salt thereof or a pharmaceutical composition including the same for use in the treatment of feline or canine cancer.

### DETAILED DESCRIPTION OF THE INVENTION

The compounds of the present invention exhibit several advantageous properties, such as favorable binding affinity to STING from various mammalian species, e.g., cat, mouse, swine and dog, especially well to dog STING, and favorable cellular activity as measured by cellular EC₅₀, i.e., in canine whole blood.

Thus, in a further aspect the invention provides new compounds of formula (I), including salts thereof, which activate STING and therefore induce cytokine production in STING-dependent fashion in vitro and/or in vivo, e.g., in dogs, and possess suitable pharmacological and pharmacokinetic properties for use in therapy, i.e., for use as medicaments.

Binding of compounds to proteins can be determined by known methods such as surface plasmon resonance, scintillation proximity assay, isothermal titration calorimetry or differential scanning fluorimetry. In the latter test, the temperature at which a protein unfolds, also called the melting temperature Tₘ, is measured by changes in fluorescence of a dye that binds to the hydrophobic parts of the protein. Tₘ shifts upon binding of a small molecule are correlated with the binding affinity of this small molecule. A high binding affinity of a STING agonist is reflected by a shift in Tₘ of >10° C., preferably >13° C., more preferably >15° C. When measured with a binding assay, the compounds in accordance with the invention preferably show an interaction with dog STING (dSTING) reflected by a shift in Tₘ of >15° C., more preferably >20° C., and even more preferably >25° C., as determined by DSF.

Since STING has been demonstrated to stimulate production of type I interferons, such as interferon beta (IFNb), in myeloid and dendritic cells, the potency of STING agonists can be evaluated in a canine whole blood (cWB) assay with IFNb secretion as a readout. In this assay, freshly collected canine blood is incubated with a STING agonist, and interferon beta levels in the supernatant are quantified by ELISA. The compounds according to the present invention typically show a cellular EC50 of below 10 μM, preferably below 5 μM, more preferably below 1 μM, most preferably below 0.5 μM. In accordance with the invention, a combination of a high dDSF and low dWB is especially favorable.

The compounds of the invention according to general formula (I)

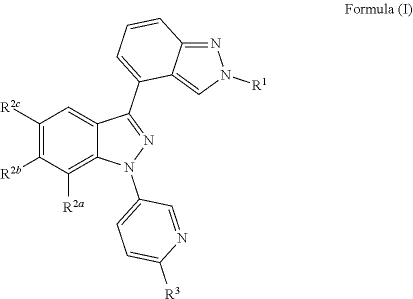

wherein
R¹ is a —C₁₋₆-alkyl or —C₃₋₆ cycloalkyl;
R^{2a} is selected from among the group consisting of —H or —C₁₋₆-alkyl;
R^{2b} is selected from among the group consisting of —H, —C₁₋₆-alkyl, —C₁₋₆-alkylene-OH, —C(O)OH, —C(O)O—C₁₋₆-alkyl and -pyrazolyl-C₁₋₆-alkyl;
R^{2c} is —H or —C₁₋₆-alkyl;
R³ is selected from among the group consisting of (* indicates the point of attachment):

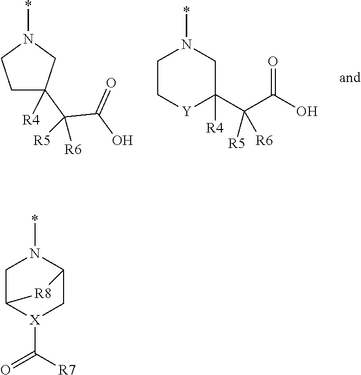

R⁴ is selected from H, —C₁₋₆-alkyl, and C₃₋₆ cycloalkyl;
R⁵ is selected from H, —C₁₋₆-alkyl, and C₃₋₆ cycloalkyl;
R⁶ is selected from H, —C₁₋₆-alkyl, and C₃₋₆ cycloalkyl;
R⁷ is selected from H, —C₁₋₆-alkyl, C₃₋₆ cycloalkyl and —OH;
R⁸ is (CH₂)ₙ, wherein n is an integer of 1-3, preferably 1 or 2;
X is CH or N; and
Y is —O—, —S—, —S(O)—, —S(O)₂—;
or a pharmaceutically acceptable salt thereof are particularly suitable for the treatment of pathophysiological processes associated with or modulated by STING, particularly for the treatment of cancer, for example feline or canine cancer or for the use as a vaccine adjuvants in, e.g. swines.

Accordingly, in another aspect, the present invention further relates to compounds of formula (I) as defined herein or pharmaceutically acceptable salts thereof or a pharmaceutical composition comprising at least one compound of formula (I) for use as a medicament.

Another aspect of the invention relates to compounds of formula (I) as defined herein or pharmaceutically acceptable salts thereof or a pharmaceutical composition comprising at least one compound of formula (I) for use in the treatment of feline or canine cancer. Other aspects of the present invention will become apparent to the person skilled in the art directly from the foregoing and following description and examples.

### Used Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general, in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached.

In case a compound of the present invention is depicted in the form of a chemical name and as a formula, in case of any discrepancy the formula shall prevail. An asterisk or a wavy line may be used in sub-formulas to indicate the bond which is connected to the core molecule as defined.

For example, the term “3-carboxypropyl-group” represents the following substituent:

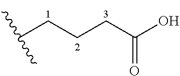 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

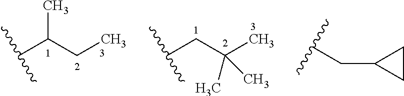

The wavy line may be used in sub-formulas to indicate the bond which is connected to the core molecule as defined. Alternatively, the asterisk may be used in sub-formulas to indicate the bond which is connected to the core molecule as defined.

1.1.1.1 Term Substituted

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

1.1.1.2 Stereochemistry-Solvates-Hydrates

Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates. Unless specifically indicated, also “pharmaceutically acceptable salts” as defined in more detail below shall encompass solvates thereof such as for instance hydrates.

1.1.1.3 Stereoisomers

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

1.1.1.4 Salts

The phrase “pharmaceutically acceptable” is employed herein to refer to those compounds, materials, compositions, and/or dosage forms which are, within the scope of sound medical judgment, suitable for use in contact with the tissues of mammals without excessive toxicity, irritation, allergic response, or other problem or complication, and commensurate with a reasonable benefit/risk ratio.

As used herein, “pharmaceutically acceptable salt” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

1.1.1.5 Halogen

The term halogen denotes fluorine, chlorine, bromine and iodine.

1.1.1.6 Heteroatoms

Heteroatoms can be present in all the possible oxidation stages. For example, sulphur can be present as sulphoxide (R—S(O)—R′) and sulphone (—R—S(O)₂—R′).

1.1.1.7 Alkyl

The term “C₁₋ₙ-alkyl”, wherein n is an integer selected from 2, 3, 4, 5 or 6, preferably 4, 5 or 6, either alone or in combination with another radical denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

1.1.1.8 Alkylene

The term “C₁₋ₙ-alkylene” wherein n is an integer selected from 2, 3, 4, 5 or 6, preferably 4, 5 or 6, either alone or in combination with another radical, denotes an acyclic, saturated, branched or linear chain divalent alkyl radical containing from 1 to n carbon atoms. For example the term C₁₋₄-alkylene includes —CH₂—, —CH₂—CH₂—, —CH(CH₃)—, —CH₂—CH₂—CH₂—, —C(CH₃)₂—, —CH(CH₂CH₃)—, —CH(CH₃)—CH₂—, —CH₂—CH(CH₃)—, —CH₂—CH₂—CH₂—CH₂—, —CH₂—CH₂—CH(CH₃)—, —CH(CH₃)—CH₂—CH₂—, —CH₂—CH(CH₃)—CH₂—, —CH₂—C(CH₃)₂—, —C(CH₃)₂—CH₂—, —CH(CH₃)—CH(CH₃)—, —CH₂—CH(CH₂CH₃)—, —CH(CH₂CH₃)—CH₂—, —CH(CH₂CH₂CH₃)—, —CH(CH(CH₃))₂— and —C(CH₃)(CH₂CH₃)—.

1.1.1.9 Alkenyl

The term “C₂₋ₘ-alkenyl” is used for a group “C₂₋ₘ-alkyl” wherein m is an integer selected from 3, 4, 5 or 6, preferably 4, 5 or 6, if at least two carbon atoms of said group are bonded to each other by a double bond.

1.1.1.10 Alkenylene

The term “C₂₋ₘ-alkenylene” is used for a group “C₂₋ₘ-alkylene” wherein m is an integer selected from 3, 4, 5 or 6, preferably 4, 5 or 6, if at least two carbon atoms of said group are bonded to each other by a double bond.

1.1.1.11 Alkynyl

The term “C₂₋ₘ-alkynyl” is used for a group “C₂₋ₘ-alkyl” wherein m is an integer selected from 3, 4, 5 or 6, preferably 4, 5 or 6, if at least two carbon atoms of said group are bonded to each other by a triple bond.

1.1.1.12 Alkynylene

The term “C₂₋ₘ-alkynylene” is used for a group “C₂₋ₘ-alkylene” wherein m is an integer selected from 3, 4, 5 or 6, preferably 4, 5 or 6, if at least two of those carbon atoms of said group are bonded to each other by a triple bond.

1.1.1.13 Cycloalkyl

The term “C₃₋ₖ-cycloalkyl”, wherein k is an integer selected from 3, 4, 5, 6, 7 or 8, preferably 4, 5 or 6, either alone or in combination with another radical denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

1.1.1.14 Cycloalkenyl

The term “C₃₋ₖ-cycloalkenyl”, wherein k is an integer integer selected from 3, 4, 5, 6, 7 or 8, preferably 4, 5 or 6, either alone or in combination with another radical, denotes a cyclic, unsaturated, but non-aromatic, unbranched hydrocarbon radical with 3 to k C atoms, at least two of which are bonded to each other by a double bond. For example the term C₃₋₇-cycloalkenyl includes cyclopropenyl, cyclobutenyl, cyclopentenyl, cyclopentadienyl, cyclohexenyl, cyclohexadienyl, cycloheptenyl cycloheptadienyl and cycloheptatrienyl.

1.1.1.15 Halo-(alkyl, alkylene or cycloalkyl)

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—.

1.1.1.16 Carbocyclyl

The term “carbocyclyl”, either alone or in combination with another radical, means a mono-, bi- or tricyclic ring structure consisting of 3 to 14 carbon atoms. The term “carbocyclyl” refers to fully saturated, partially saturated and aromatic ring systems. The term “carbocyclyl” encompasses fused, bridged and spirocyclic systems.

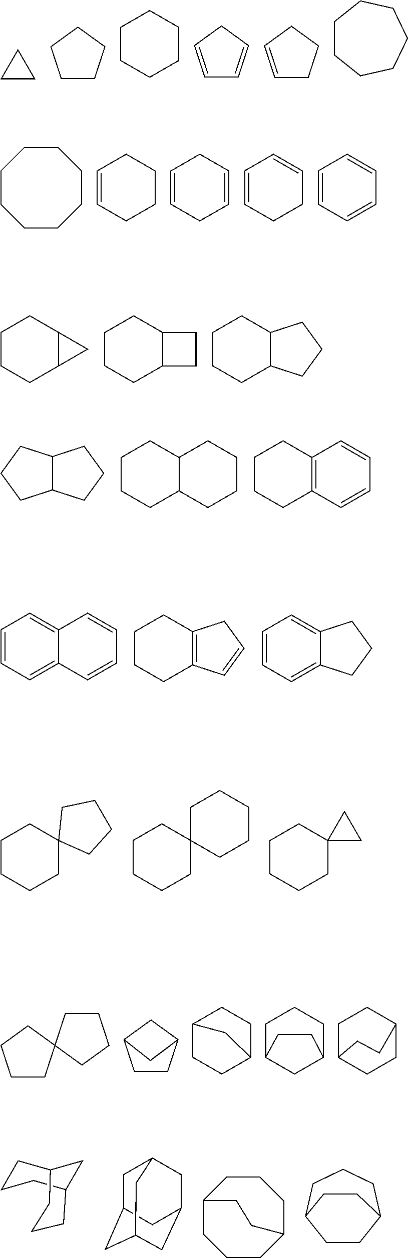

1.1.1.17 Aryl

The term “aryl” as used herein, either alone or in combination with another radical, denotes a carbocyclic aromatic monocyclic group containing 6 carbon atoms which is optionally further fused to a second five- or six-membered, carbocyclic group which is aromatic, saturated or unsaturated. Aryl includes, but is not limited to, phenyl, indanyl, indenyl, naphthyl, anthracenyl, phenanthrenyl, tetrahydronaphthyl and dihydronaphthyl.

1.1.1.18 Heterocyclyl

The term “heterocyclyl” means a saturated or unsaturated mono- or polycyclic ring system optionally comprising aromatic rings, containing one or more heteroatoms selected from N, O, S, SO, SO₂, consisting of 3 to 14 ring atoms wherein none of the heteroatoms is part of the aromatic ring. The term “heterocyclyl” is intended to include all the possible isomeric forms.

Thus, the term “heterocyclyl” includes the following exemplary structures (not depicted as radicals as each form is optionally attached through a covalent bond to any atom so long as appropriate valences are maintained):

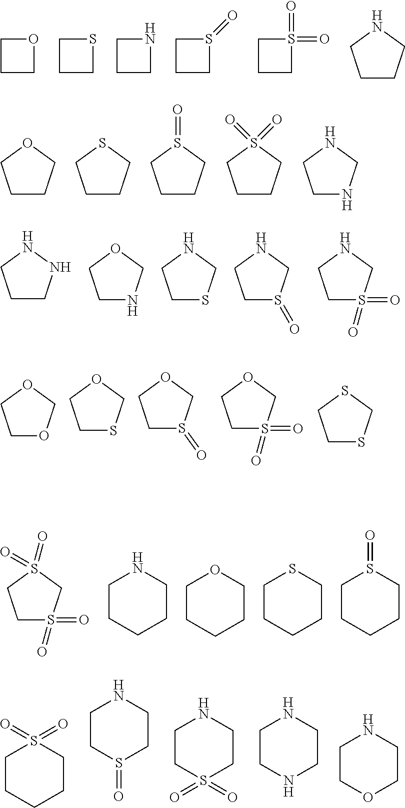

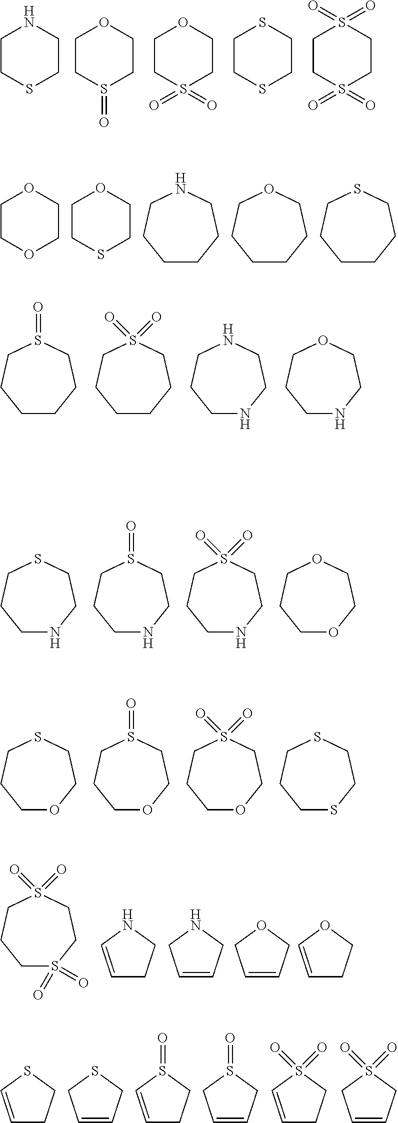

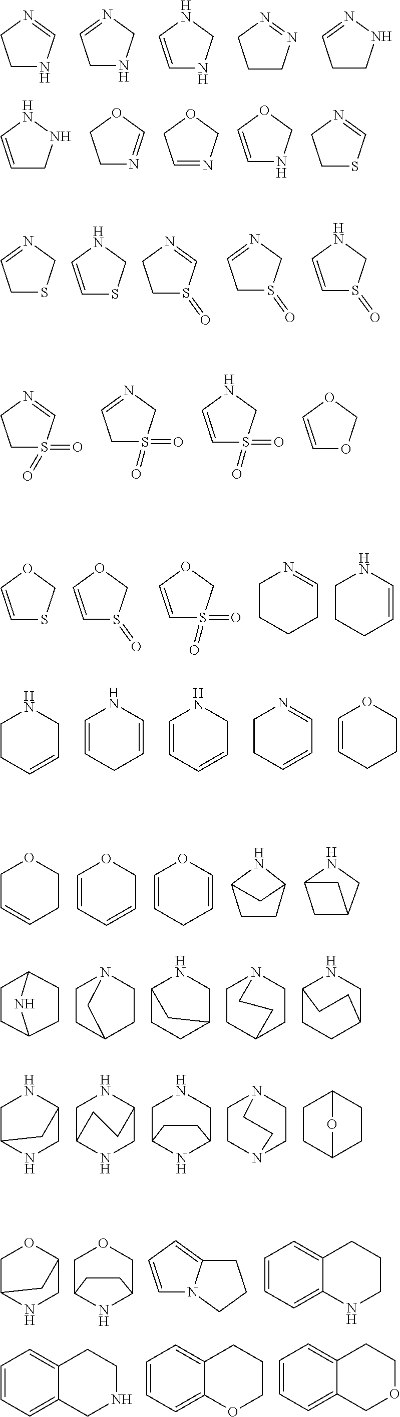

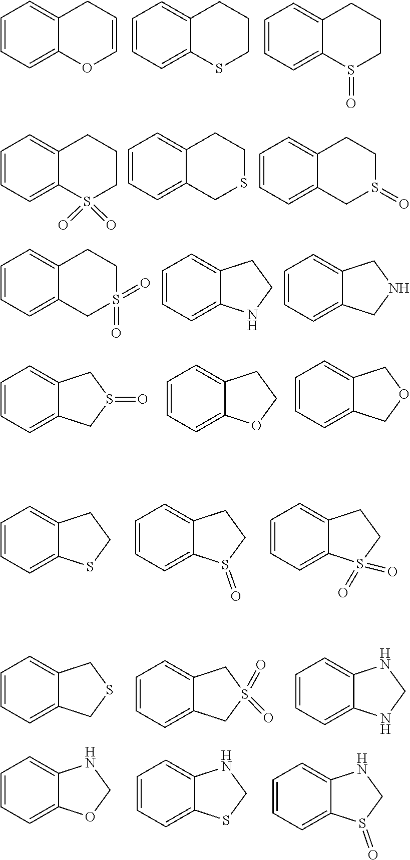

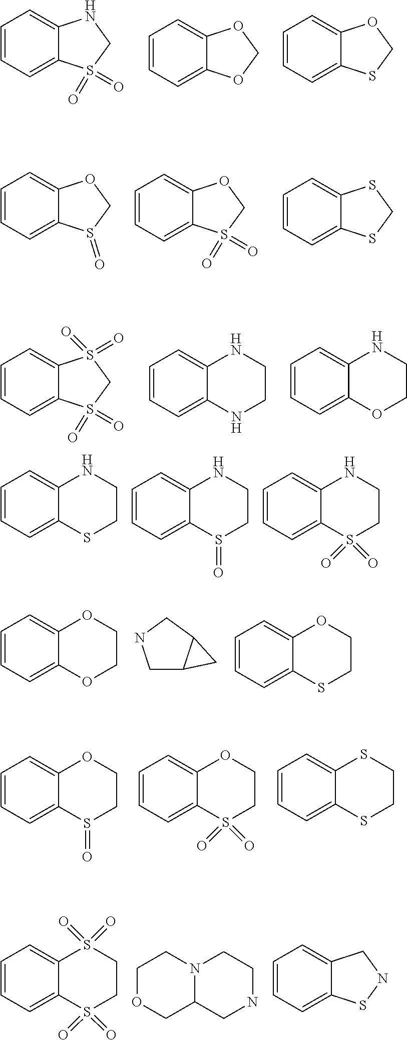

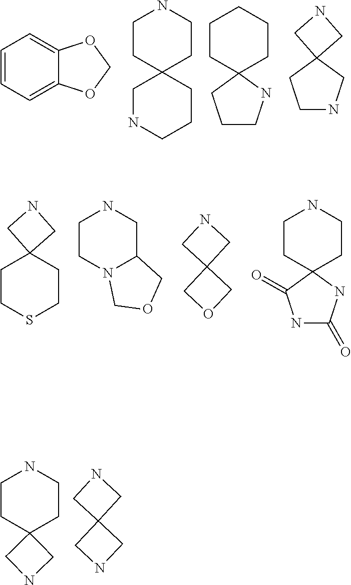

1.1.1.19 Heteroaryl

The term “heteroaryl” means a mono- or polycyclic-ring system, comprising at least one aromatic ring, containing one or more heteroatoms selected from N, O, S, SO or SO₂, consisting of 5 to 14 ring atoms wherein at least one of the heteroatoms is part of an aromatic ring, wherein the resulting ring system must be chemically stable. The term “heteroaryl” is intended to include all the possible isomeric forms.

Thus, the term “heteroaryl” includes the following exemplary structures (not depicted as radicals as each form is optionally attached through a covalent bond to any atom so long as appropriate valences are maintained):

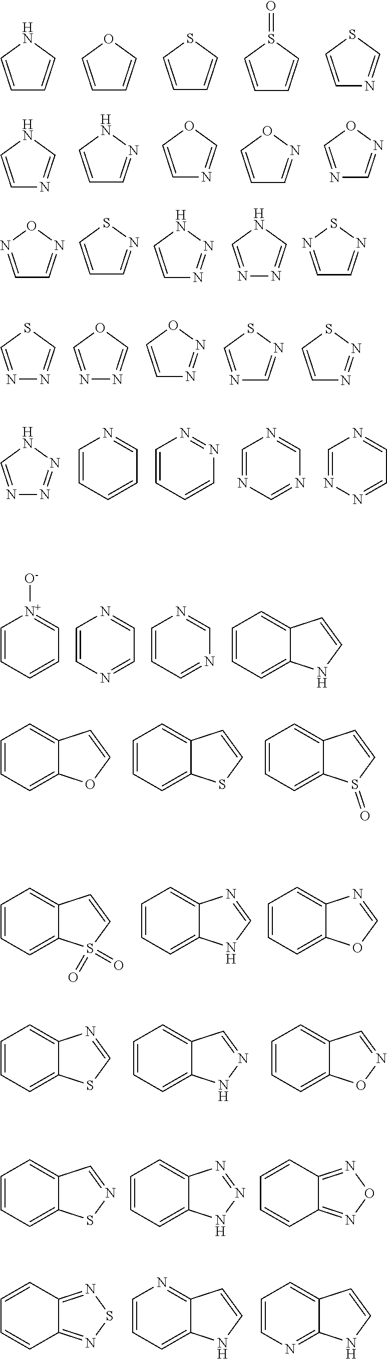

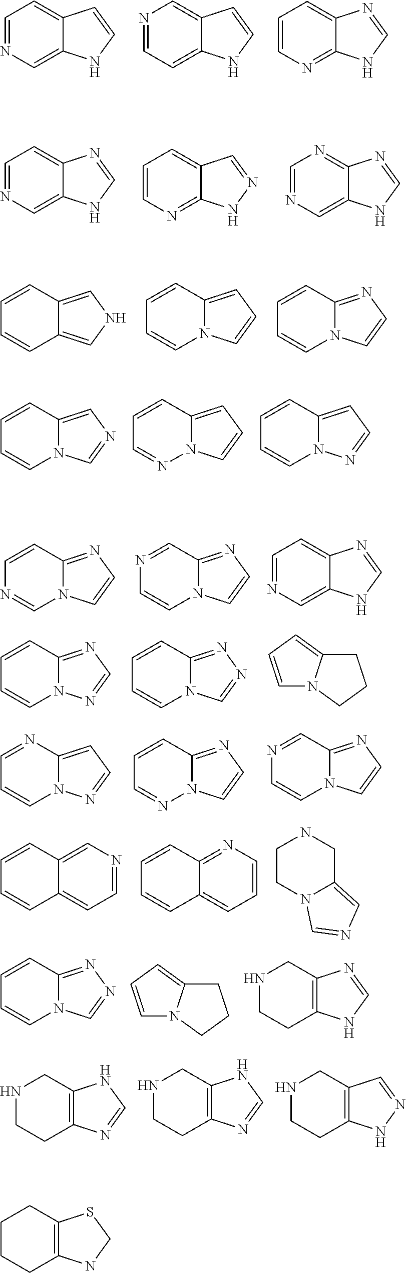

Many of the terms given above may be used repeatedly in the definition of a formula or group and in each case have one of the meanings given above, independently of one another.

The term “bicyclic ring systems” means groups consisting of 2 joined cyclic substructures including spirocyclic, fused, and bridged ring systems.

### PREFERRED EMBODIMENTS

One preferred embodiment of the invention relates to a compound of formula (I)

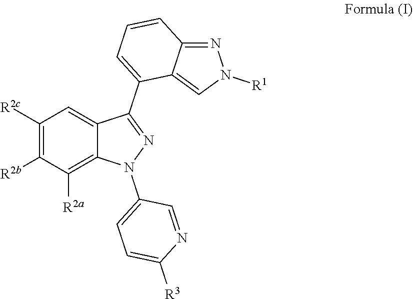

or a pharmaceutically acceptable salt thereof, wherein
R¹ is a —C₁₋₆-alkyl or —C₃₋₆ cycloalkyl;
R^{2a} is selected from among the group consisting of —H or —C₁₋₆-alkyl;
R^{2b} is selected from among the group consisting of —H, —C₁₋₆-alkyl, —C₁₋₆-alkylene-OH, —C(O)OH, —C(O)O—C₁₋₆-alkyl and -pyrazolyl-C₁₋₆-alkyl;
R^{2c} is —H or —C₁₋₆-alkyl;
R³ is selected from among the group consisting of (* indicates the point of attachment):

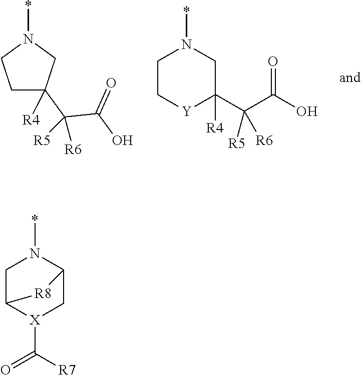

R⁴ is selected from H, —C₁₋₆-alkyl, and C₃₋₆ cycloalkyl;
R⁵ is selected from H, —C₁₋₆-alkyl, and C₃₋₆ cycloalkyl;
R⁶ is selected from H, —C₁₋₆-alkyl, and C₃₋₆ cycloalkyl;
R⁷ is selected from H, —C₁₋₆-alkyl, C₃₋₆ cycloalkyl and —OH;
R⁸ is (CH₂)ₙ, wherein n is an integer of 1-3, preferably 1 or 2;
X is CH or N; and if X is N, then R⁷ is not —OH; and
Y is —O—, —S—, —S(O)—, —S(O)₂—.

Another particularly preferred embodiment relates to a compound of formula (I) or a pharmaceutically acceptable salt thereof, wherein R¹ is —C₁₋₆-alkyl, preferably methyl.

Another particularly preferred embodiment relates to a compound of formula (I) or a pharmaceutically acceptable salt thereof, wherein R^{2a} is —C₁₋₆-alkyl, preferably methyl; R^{2b} is —H or —C₁₋₆-alkyl; and R^{2c} is —H or —C₁₋₆-alkyl.

Another particularly preferred embodiment relates to a compound of formula (I) or a pharmaceutically acceptable salt thereof, wherein R¹ is —C₁₋₆-alkyl, preferably methyl, R^{2a} is —C₁₋₆-alkyl, preferably methyl; R^{2b} is —H or —C₁₋₆-alkyl; and R^{2c} is —H or —C₁₋₆-alkyl.

Another particularly preferred embodiment relates to a compound of formula (I) or a pharmaceutically acceptable salt thereof, wherein R^{2a} is methyl; R^{2b} is —H; and R^{2c} is —H.

Another particularly preferred embodiment relates to a compound of formula (I) or a pharmaceutically acceptable salt thereof, wherein R¹ is —C₁₋₆-alkyl, preferably methyl, R^{2a} is methyl; R^{2b} is —H; and R^{2c} is —H.

Another particularly preferred embodiment relates to a compound of formula (I) or a pharmaceutically acceptable salt thereof, wherein R³ is

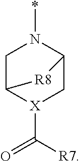

Another particularly preferred embodiment relates to a compound of formula (I) or a pharmaceutically acceptable salt thereof, wherein R³ is

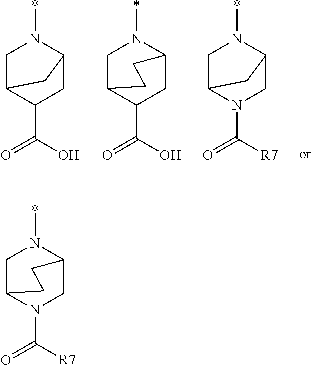

wherein R⁷ is —C₁₋₆-alkyl, preferably methyl.

Another particularly preferred embodiment relates to a compound of formula (I) or a pharmaceutically acceptable salt thereof, wherein R³ is

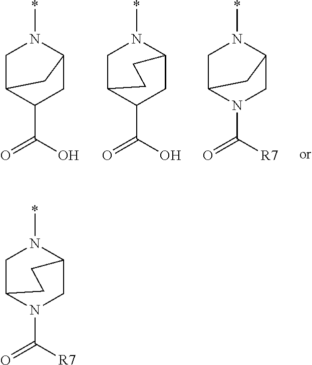

and R¹ is —C₁₋₆-alkyl, preferably methyl.

Another particularly preferred embodiment relates to a compound of formula (I) or a pharmaceutically acceptable salt thereof, wherein R³ is

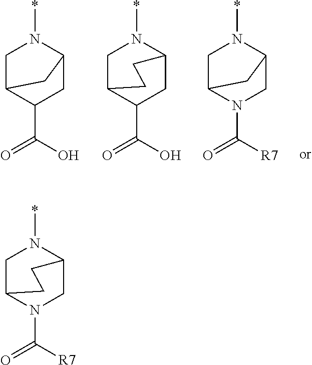

and R^{2a} is —C₁₋₆-alkyl, preferably methyl; R^{2b} is —H or —C₁₋₆-alkyl; and R^{2c} is —H or —C₁₋₆-alkyl.

Another particularly preferred embodiment relates to a compound of formula (I) or a pharmaceutically acceptable salt thereof, wherein R³ is

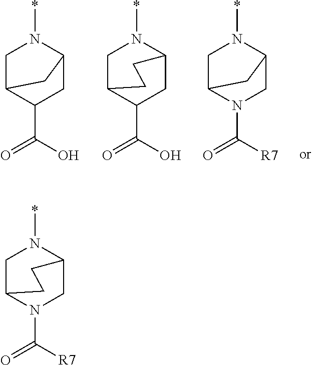

R¹ is —C₁₋₆-alkyl, preferably methyl; R^{2a} is —C₁₋₆-alkyl, preferably methyl; R^{2b} is —H or —C₁₋₆-alkyl; and R^{2c} is —H or —C₁₋₆-alkyl.

Another particularly preferred embodiment relates to a compound of formula (I) or a pharmaceutically acceptable salt thereof, wherein R³ is

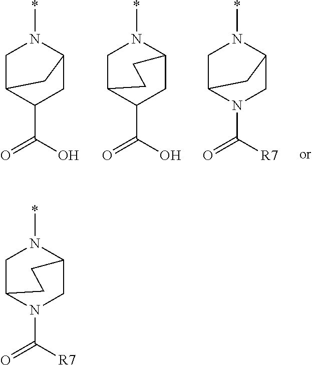

and R¹ is —C₁₋₆-alkyl, preferably methyl, R^{2a} is methyl; R^{2b} is —H; and R^{2c} is —H.

Another particularly preferred embodiment relates to a compound of formula (I) or a pharmaceutically acceptable salt thereof, wherein R³ is

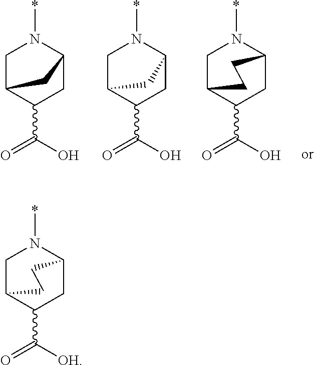

Another particularly preferred embodiment relates to a compound of formula (I) or a pharmaceutically acceptable salt thereof, wherein R³ is

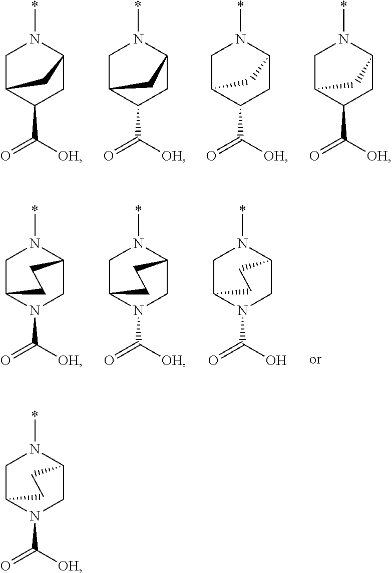

Another particularly preferred embodiment relates to a compound of formula (I) or a pharmaceutically acceptable salt thereof, wherein R³ is

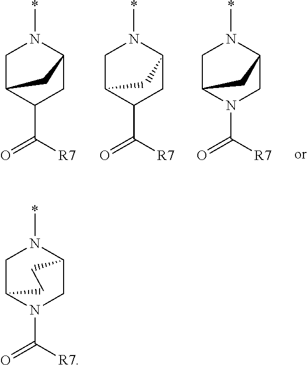

Another particularly preferred embodiment relates to a compound of formula (I) or a pharmaceutically acceptable salt thereof, wherein R³ is

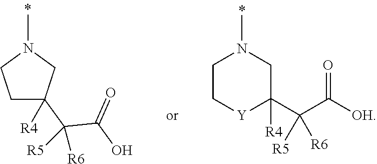

Another particularly preferred embodiment relates to a compound of formula (I) or a pharmaceutically acceptable salt thereof, wherein R³ is

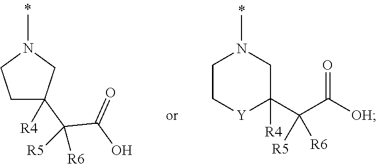

R⁴ is —H or methyl, preferably —H; R⁵ and R⁶ are independently —H or methyl, preferably R⁵ and R⁶ are both —H; and Y is O.

Another particularly preferred embodiment relates to a compound of formula (I) or a pharmaceutically acceptable salt thereof, wherein R³ is

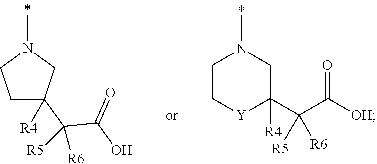

R¹ is methyl; R⁴ is —H or methyl, preferably —H; R⁵ and R⁶ are independently —H or methyl, preferably R⁵ and R⁶ are both —H; Y is O.

Another particularly preferred embodiment relates to a compound of formula (I) or a pharmaceutically acceptable salt thereof, wherein R³ is

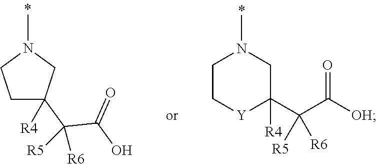

R^{2a} is —C₁₋₆-alkyl, preferably methyl; R^{2b} is —H or —C₁₋₆-alkyl; and R^{2c} is —H or —C₁₋₆-alkyl; R⁴ is —H or methyl, preferably —H; R⁵ and R⁶ are independently —H or methyl, preferably R⁵ and R⁶ are both —H; Y is O.

Another particularly preferred embodiment relates to a compound of formula (I) or a pharmaceutically acceptable salt thereof, wherein R³ is

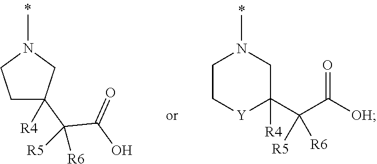

R¹ is methyl; R^{2a} is —C₁₋₆-alkyl, preferably methyl; R^{2b} is —H or —C₁₋₆-alkyl; and R^{2c} is —H or —C₁₋₆-alkyl; R⁴ is —H or methyl, preferably —H; R⁵ and R⁶ are independently —H or methyl, preferably R⁵ and R⁶ are both —H; Y is O.

Another particularly preferred embodiment relates to a compound of formula (I) or a pharmaceutically acceptable salt thereof, wherein R¹ is methyl; R^{2a} is methyl; R^{2b} and R^{2c} are —H; R³ is

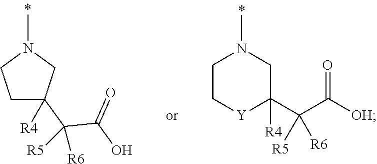

R⁴, R⁵ and R⁶ are —H; and Y is O.

Particularly preferred compounds of the invention are selected from the following structures:

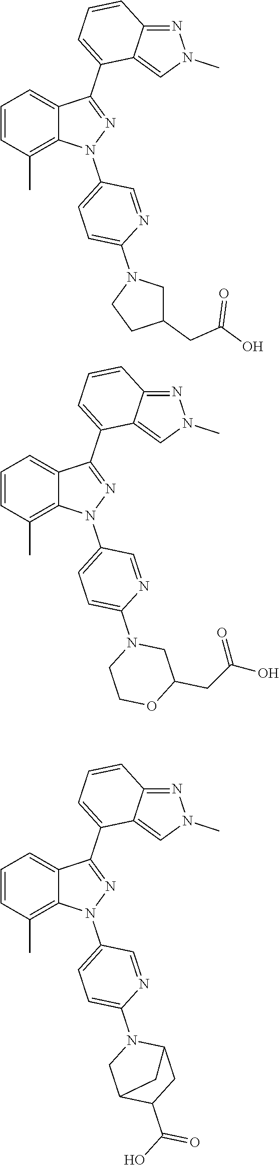

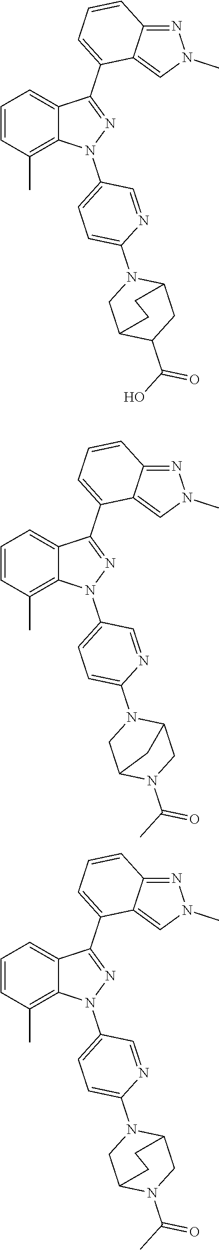

or a pharmaceutically acceptable salt thereof.

Further particularly preferred compounds of the invention are:

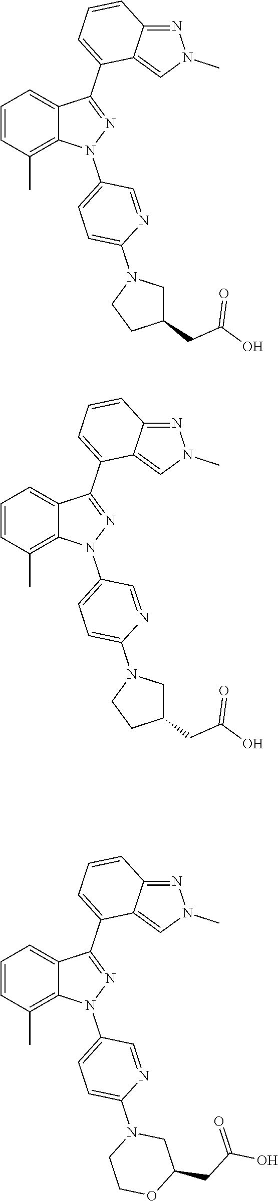 [SMILES: Cc1cccc2c(-c3cccc4nn(C)cc34)nn(-c3ccc(N4CC[C@H](CC(=O)O)C4)nc3)c12 ; Cc1cccc2c(-c3cccc4nn(C)cc34)nn(-c3ccc(N4CC[C@@H](CC(=O)O)C4)nc3)c12 ; Cc1cccc2c(-c3cccc4nn(C)cc34)nn(-c3ccc(N4CCO[C@H](CC(=O)O)C4)nc3)c12]

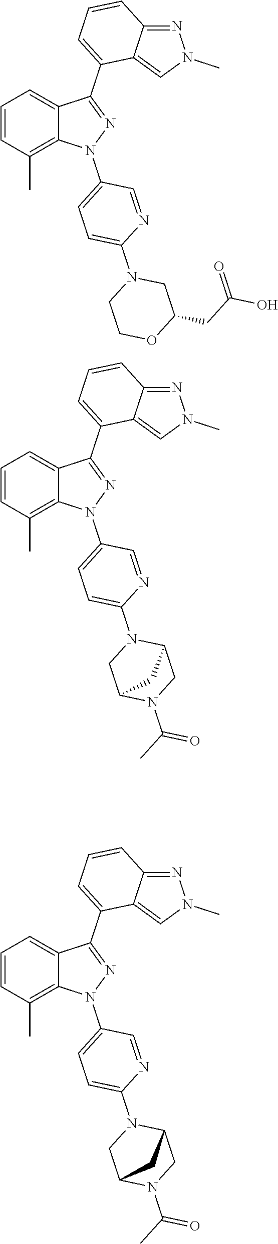

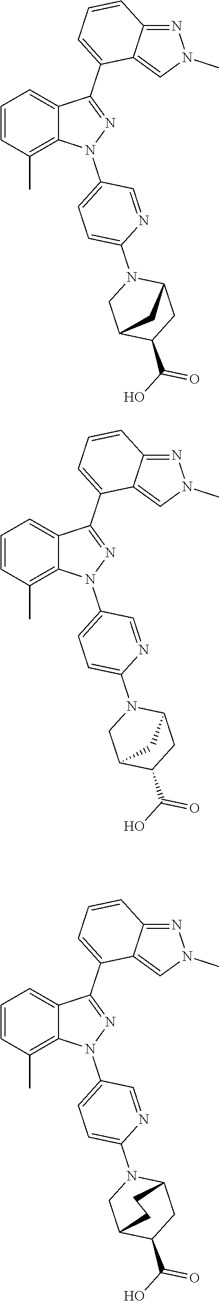

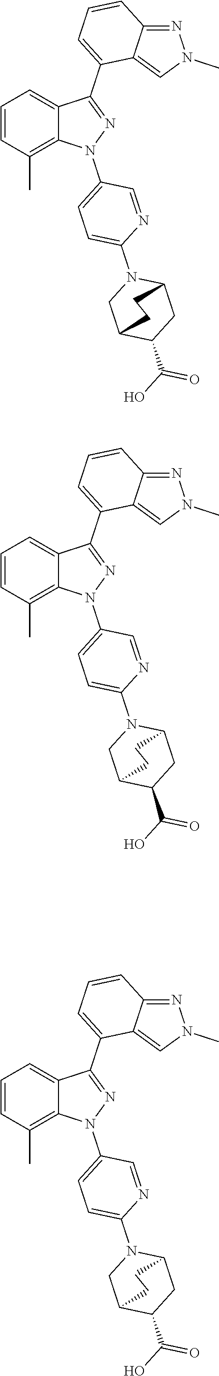 [SMILES: Cc1cccc2c(-c3cccc4nn(C)cc34)nn(-c3ccc(N4C[C@@H]5CC[C@H]4C[C@@H]5C(=O)O)nc3)c12 ; Cc1cccc2c(-c3cccc4nn(C)cc34)nn(-c3ccc(N4C[C@H]5CC[C@@H]4C[C@H]5C(=O)O)nc3)c12 ; Cc1cccc2c(-c3cccc4nn(C)cc34)nn(-c3ccc(N4C[C@H]5CC[C@@H]4C[C@@H]5C(=O)O)nc3)c12]

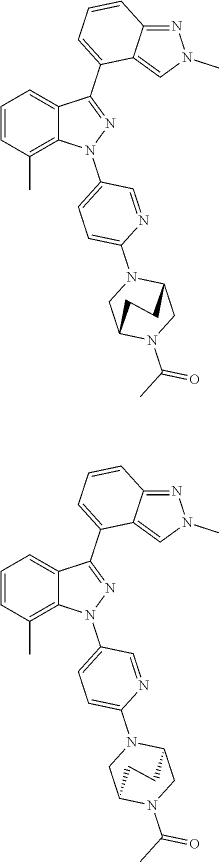 [SMILES: CC(=O)N1C[C@@H]2CC[C@H]1CN2c1ccc(-n2nc(-c3cccc4nn(C)cc34)c3cccc(C)c32)cn1 ; CC(=O)N1C[C@H]2CC[C@@H]1CN2c1ccc(-n2nc(-c3cccc4nn(C)cc34)c3cccc(C)c32)cn1]

or a pharmaceutically acceptable salt thereof.

In one embodiment, the invention relates to compounds of formula (I) in their salt free forms. In another embodiment, the invention relates to compounds of formula (I) in form of pharmaceutically acceptable salts.

Any and each of the definitions of R¹, R^{2a}, R^{2b}, R^{2c}, R³, R⁴, R⁶, R⁶, R⁷, R⁸, X and Y identified above for formula (I) may be combined with each other.

In one aspect, the invention relates to a pharmaceutical composition comprising at least one compound according to formula (I) or a pharmaceutically acceptable salt thereof and a pharmaceutically acceptable carrier.

It is found that compounds of formula (I) or pharmaceutically acceptable salts thereof may be useful in the prevention and/or for the treatment of diseases and/or conditions wherein the modulation of STING is of therapeutic benefit. Thus, in another aspect, the invention relates to a compound of formula (I) or a pharmaceutically acceptable salt thereof or a pharmaceutical composition comprising at least one compound of formula (I) for use as a medicament. In one aspect, the invention relates to compounds of formula (I),a pharmaceutically acceptable salt thereof or a pharmaceutical composition comprising at least one of these compounds for use in the treatment of feline or canine cancer.

In another aspect, the compounds in accordance with the invention show an interaction with dog STING (dSTING) reflected by a shift in Tₘ of >15° C., more preferably >20° C., and even more preferably >25° C., as determined by DSF.

In a preferred embodiment, the compounds in accordance with the present invention show an interaction with dSTING, as determined by DSF (dDSF) and also induce cytokine secretion in dog whole blood (dWB).

In a more preferred embodiment, the compounds in accordance with the invention show a combination of a high dDSF and low dWB.

### METHOD OF TREATMENT

In one aspect the invention relates to the use of a compound of formula (I), a pharmaceutically acceptable salt thereof or a pharmaceutical composition comprising at least one of these compounds in a method of treating a disease.

In particular, the compounds of general formula (I) or salts thereof are useful in the prevention and/or for the treatment of diseases and/or conditions in mammals, for example in cats, mice, swines and dogs, wherein the modulation of STING is of therapeutic benefit. Furthermore, due to their activity the compounds of the present invention are suitable as vaccine adjuvants.

Diseases and conditions associated with or modulated by STING embrace, but are not limited to inflammation, allergic or autoimmune diseases, for example allergic rhinitis or asthma, infectious diseases or cancer.

Autoimmune diseases include, but are not limited to systemic lupus erythematosus, psoriasis, insulin-dependent diabetes mellitus (IDDM), dermatomyositis and Sjogren's syndrome (SS).

The compounds of the invention may be used to treat inflammation of any tissue and organs of the body, including but not limited to musculoskeletal inflammation, vascular inflammation, neural inflammation, digestive system inflammation, ocular inflammation, inflammation of the reproductive system, and other inflammation.

Examples of musculoskeletal inflammation which may be treated with compounds of the invention include arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic). Examples of ocular inflammation which may be treated with the compounds of the invention include blepharitis, blepharochalasis, conjunctivitis, dacryoadenitis, keratitis, keratoconjunctivitis sicca (dry eye), scleritis, trichiasis, and uveitis.

Examples of inflammation of the nervous system which may be treated with the compounds of the invention include encephalitis, Guillain-Barre syndrome, meningitis, neuromyotonia, narcolepsy, multiple sclerosis, myelitis and schizophrenia.

Examples of inflammation of the vasculature or lymphatic system which may be treated with the compounds of the invention include arthrosclerosis, arthritis, phlebitis, vasculitis, and lymphangitis. Examples of inflammatory conditions of the digestive system which may be treated with the compounds of the invention include cholangitis, cholecystitis, enteritis, enterocolitis, gastritis, gastroenteritis, inflammatory bowel disease (such as Crohn's disease and ulcerative colitis), ileitis, and proctitis.

Examples of inflammatory conditions of the reproductive system which may be treated with the compounds of the invention include cervicitis, chorioamnionitis, endometritis, epididymitis, omphalitis, oophoritis, orchitis, salpingitis, tubo-ovarian abscess, urethritis, vaginitis, vulvitis, and vulvodynia.

The compounds may be used to treat autoimmune conditions having an inflammatory component. Such conditions include acute disseminated alopecia universalis, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, Goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, lyme disease, morphea, psoriasis, sarcoidosis, scleroderma, ulcerative colitis, and vitiligo.

The compounds may be used to treat T-cell mediated hypersensitivity diseases having an inflammatory component. Such conditions include contact hypersensitivity, contact dermatitis (including that due to poison ivy), urticaria, skin allergies, respiratory allergies (hayfever, allergic rhinitis) and gluten-sensitive enteropathy (Celiac disease).

Other inflammatory conditions which may be treated with the compounds include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatitis, transplant rejection (involving organs such as kidney, liver, heart, lung, pancreas (e.g., islet cells), bone marrow, cornea, small bowel, skin allografts, skin homografts, and heart valve xenografts, serum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis.

Preferred treatments include treatment of transplant rejection, rheumatoid arthritis, psoriatic arthritis, multiple sclerosis. Type 1 diabetes, asthma, inflammatory bowel disease, systemic lupus erythematosis, psoriasis, chronic pulmonary disease, and inflammation accompanying infectious conditions (e.g., sepsis).

In one aspect the disease or condition to be treated using compounds of the invention is cancer. Examples of cancer diseases and conditions in which compounds of formula (I), or salts or solvates thereof may have potentially beneficial anti-tumor effects include, but are not limited to, cancers of the lung, bone, pancreas, skin, brain, head, neck, uterus, ovaries, stomach, colon, colorectal, breast, esophagus, small intestine, bowel, endocrine system, thyroid gland, parathyroid gland, adrenal gland, urethra, prostate, penis, testes, ureter, bladder, kidney or liver, bile duct; urothelial cancer; rectal cancer; cancer of the anal region; carcinomas of the fallopian tubes, endometrium, cervix, vagina, vulva, renal pelvis, renal cell; sarcoma; sarcoma of soft tissue; myxoma; rhabdomyoma; fibroma; lipoma; teratoma; cholangiocarcinoma; hepatoblastoma; angiosarcoma; hemagioma; hepatoma; fibrosarcoma; chondrosarcoma; myeloma; chronic or acute leukemia; lymphocytic lymphomas; primary CNS lymphoma; neoplasms of the CNS; spinal axis tumours; squamous cell carcinomas; synovial sarcoma; malignant pleural mesotheliomas; brain stem glioma; pituitary adenoma; bronchial adenoma; chondromatous hanlartoma; mesothelioma; Hodgkin's Disease or a combination of one or more of the foregoing cancers.

Preferred cancers, which may be treated with compounds according to the invention, are skin, lung, e.g. small-cell lung cancer, non-small cell lung cancer, liver, pancreas, colon, colorectal, brain, breast, ovary, prostate, kidney, bladder, bile duct, endometrium, thyroid gland, cervix, stomach, head, neck, sarcoma, sarcoma of soft tissue, esophagus, head-and-neck-cancer, rectal and urothelial cancer, as well as lymphoma.

The new compounds may be used for the prevention, palliative, curative or semi-curative, short-term or long-term treatment of the above-mentioned diseases, optionally also in combination with surgery, radiotherapy or other “state-of-the-art” compounds, such as e.g. cytostatic or cytotoxic substances, cell proliferation inhibitors, anti-angiogenic substances, steroids, antibodies, nanobodies, cancer-targeting agents, viruses, including but not limited to oncolytic viruses, or immunogenic cell death inducers.

The new compounds may also be used for the prevention, palliative, curative or semi-curative, short-term or long-term treatment of the above-mentioned diseases by combining different administration routes, e.g. intravenous, intratumoral, subcutaneous, inhalative, oral etc. for the compounds, optionally also in combination with surgery, radiotherapy or other “state-of-the-art” compounds, such as e.g. cytostatic or cytotoxic substances, cell proliferation inhibitors, anti-angiogenic substances, steroids, antibodies, nanobodies, cancer-targeting agents, viruses, including but not limited to oncolytic viruses, or immunogenic cell death inducers. Merely as an example of surgery, partial or complete tumor excision may be combined with the compounds of the invention. Merely as an example of radiotherapy, external beam radiotherapy may be combined with the compounds of the invention.

In their role as adjuvants, in certain embodiments the present compounds and compositions may be used as adjuvants in a therapeutic or prophylactic strategy employing vaccine(s). Thus, the compounds of the present invention, or salts thereof, may be used together with one or more vaccines selected to stimulate an immune response to one or more predetermined antigens. The compounds of the present invention, or salts thereof, may be provided together with, or in addition to, such vaccines.

Such vaccine(s) can comprise inactivated or attenuated bacteria or viruses comprising the antigens of interest, purified antigens, live viral or bacterial delivery vectors recombinantly engineered to express and/or secrete the antigens, antigen presenting cell (APC) vectors comprising cells that are loaded with the antigens or transfected with a composition comprising a nucleic acid encoding the antigens, liposomal antigen delivery vehicles, or naked nucleic acid vectors encoding the antigens. This list is not meant to be limiting. By way of example, such vaccine(s) may also comprise an inactivated tumor cell or an oncolytic virus that expresses and secretes one or more of GM-CSF, CCL20, CCL3, IL-12p70, FLT-3 ligand, cytokines.

Accordingly, the present invention relates to a compound of general formula (I) for use as a medicament, e.g., for treating feline or canine cancer, or as vaccine adjuvants for use in, e.g., swines.

In a preferred aspect, the invention relates to a compound of formula (I), a pharmaceutically acceptable salt thereof or a pharmaceutical composition comprising at least one compound of formula (I) for use in the treatment of feline or canine cancer.

In one embodiment, the invention relates to the compound or a pharmaceutically acceptable salt thereof or a pharmaceutical composition comprising at least one of the compounds for use in the treatment of canine cancer, wherein the canine cancer is selected from osteosarcoma (OSA), oral melanoma, B-cell lymphoma, urothelial carcinoma (UC), hemangiosarcoma, mast cell tumor, soft tissue sarcoma, squamous cell carcinoma, T-cell lymphoma, mammary gland adenocarcinoma and anal sac carcinoma.

In another embodiment, the invention relates to the compound or a pharmaceutically acceptable salt thereof or a pharmaceutical composition comprising at least one of the compounds for use in the treatment of feline cancer, wherein the feline cancer is selected from B-cell and/or T-cell lymphoma, squamous cell carcinoma, mammary gland adenocarcinoma, mast cell tumors and injection site sarcoma.

In a further aspect the present invention relates to a method of treatment and/or prevention of the above-mentioned diseases and conditions which comprises administering to a subject an effective amount of a compound of formula (I), a pharmaceutically acceptable salt thereof or a pharmaceutical composition comprising at least one compound of formula (I).

In a particularly preferred embodiment, the present invention relates to a method of treating feline or canine cancer which comprises administering an effective amount of a compound of formula (I), a pharmaceutically acceptable salt thereof or a pharmaceutical composition comprising at least one compound of formula (I) to a feline or canine.

In a more preferred embodiment, the present invention relates to a method of treating canine cancer which comprises administering an effective amount of a compound of formula (I), a pharmaceutically acceptable salt thereof or a pharmaceutical composition comprising at least one compound of formula (I) to a canine, wherein the canine cancer is selected from canine cancer is selected from osteosarcoma (OSA), oral melanoma, B-cell lymphoma, urothelial carcinoma (UC), hemangiosarcoma, mast cell tumor, soft tissue sarcoma, squamous cell carcinoma, T-cell lymphoma, mammary gland adenocarcinoma and anal sac carcinoma.

In another more preferred embodiment, the present invention relates to a method of treating feline cancer which comprises administering an effective amount of a compound of formula (I), a pharmaceutically acceptable salt thereof or a pharmaceutical composition comprising at least one compound of formula (I) to a feline, wherein the feline cancer is selected from B-cell and/or T-cell lymphoma, squamous cell carcinoma, mammary gland adenocarcinoma, mast cell tumors and injection site sarcoma.

In a further aspect, the present invention relates to a compound of general formula (I) for use in the treatment and/or prevention of above-mentioned cancers, before or after tumor excision and/or radiotherapy.

In a further aspect, the present invention relates to the use of a compound of general formula (I) for the preparation of a medicament for the treatment and/or prevention of above-mentioned diseases and conditions.

In another aspect, the present invention relates to a method of treating canine or feline cancer which comprises administering an effective amount of a compound of formula (I), a pharmaceutically acceptable salt thereof or a pharmaceutical composition comprising at least one compound of formula (I) to a canine or feline in combination with radiotherapy.

Pharmaceutical Compositions

In another aspect of the present invention, pharmaceutical compositions comprising at least one of the above-mentioned compounds are provided.

The pharmaceutical compositions may be formulated in a way that they are suitable for the administration of therapeutically effective amounts of said compounds. Suitable preparations for administering the compounds of formula (I) will be apparent to those with ordinary skill in the art and include for example tablets, pills, capsules, suppositories, lozenges, troches, solutions, syrups, elixirs, sachets, injectable solutions (subcutaneously, intravenously, intramuscularly, intra-peritoneal, intra-tumorally and peri-tumorally), inhalables, infusions, elixirs, emulsions, and powders. Furthermore, the compounds according to the invention may be administered via targeted delivery platforms, for example such targeted delivery platforms may be antibody-drug conjugates, nanobody-drug conjugates, peptide-drug conjugates, virus-like particles, or nanoparticle formulations.

Suitable tablets may be obtained, for example, by mixing one or more compounds according to formula I with known excipients, for example inert diluents, carriers, disintegrants, adjuvants, surfactants, binders and/or lubricants.

For the purposes of this disclosure, the pharmaceutical compositions may be administered by a variety of means, including non-parenterally, parenterally, by inhalation spray, topically, nasally, orally, or rectally in formulations containing pharmaceutically acceptable carriers, adjuvants and vehicles. The pharmaceutical compositions of the disclosure may be administered in the form of a sterile injectable preparation, such as a sterile injectable aqueous or oleaginous suspension.

Combination Therapy

The compounds of the invention may be used on their own or may be combined with one or more further therapeutic agent(s).

In a further aspect, the invention provides a method of treatment of a disease or condition in which modulation of STING is beneficial comprising administering a therapeutically effective amount of a combination comprising a compound of formula (I), or a pharmaceutically acceptable salt thereof, and at least one further therapeutic agent.

In a further aspect, the invention provides a method of treatment of inflammation, allergic or autoimmune diseases, infectious diseases or cancer comprising administering a therapeutically effective amount of a combination comprising a compound of formula (I), or a pharmaceutically acceptable salt thereof, and at least one further therapeutic agent.

The actual pharmaceutically effective amount or therapeutic dosage will of course depend on factors known by those skilled in the art such as age and weight of the patient, route of administration and severity of disease. In any case the combination will be administered at dosages and in a manner which allows a pharmaceutically effective amount to be delivered based upon patient's unique condition.

In certain embodiments, the compounds and compositions thereof described herein are administered in conjunction with one or more additional compositions including vaccines intended to stimulate an immune response to one or more predetermined antigens; adjuvants; CTLA-4 and PD-1 pathway antagonists, lipids, liposomes, chemotherapeutic agents, immunomodulatory cell lines, cancer-targeting agents, immunogenic cell-death inducers, immuno-modulating agents, wherein the immunomodulating agents may be understood as agents of a general activation-modulation type in general as well as agents modulating and/or increasing the frequency of a certain immune cell subtype, etc.

The compounds and compositions thereof described herein may be administered before, after, and/or simultaneously with an additional therapeutic or prophylactic composition or modality.

The compounds, compositions, including any combinations with one or more additional therapeutic agent(s), according to the invention may be administered by mucosal (e.g. oral, sublingual, vaginal, nasal, cervical, etc.), intra-tumoral, intra-peritoneal, peri-tumoral, transdermal, inhalative, or parenteral (e.g. subcutaneous, intravenous, intramuscular, intraarterial, intradermal, intrathecal and epidural administrations) route.

Furthermore, the compounds, compositions, including any combinations with one or more additional therapeutic agents, according to the invention may be administered via targeted delivery platforms, for example such targeted delivery platforms can be antibody-drug conjugates, nanobody-drug conjugates, peptide-drug conjugates, virus-like particles, or nanoparticles.

Of the possible methods of administration, intra-peritoneal, intra-tumoral, peri-tumoral, subcutaneous, inhalative or intravenous administration is preferred. The compounds, compositions, including any combinations with one or more additional therapeutic agents, according to the invention may also be administered before, after, and/or simultaneously by a combination of different methods of administration. Simply by way of an example, an inhalative or intravenous administration may be followed by an intra-tumoral or peri-tumoral administration or an intra-tumoral or peri-tumoral administration may be followed by an inhalative or intravenous administration. Additionally, such an administration of the compounds via different routes may be before or after additional therapeutic step, such as tumor excision or radiotherapy.

In a particularly preferred embodiment, a compound of the invention, a pharmaceutically acceptable salt thereof or a pharmaceutical composition comprising at least one compound of the invention is used in combination with radiotherapy. Simply by way of an example, the compounds of the invention may be administered after radiotherapy. Furthermore, the compounds of the invention may be given by intravenous administration after radiotherapy. Furthermore, the compounds of the invention may be given by intravenous administration after tumor excision. Furthermore, the compounds of the invention may be given by intra-tumoral administration after radiotherapy. Furthermore, the compounds of the invention may be given by peri-tumoral administration after radiotherapy. Furthermore, the compounds of the invention may be given by inhalative administration after tumor excision. Furthermore, the compounds of the invention may be given by intravenous administration, followed by intra-tumoral administration, and both administrations take place after radiotherapy. Furthermore, the compounds of the invention may be given by intra-tumoral administration, followed by intravenous administration, and both administrations take place after radiotherapy. Furthermore, the compounds of the invention may be given by intravenous administration, followed by peri-tumoral administration, and both administrations take place after radiotherapy. Furthermore, the compounds of the invention may be given by peri-tumoral administration, followed by intravenous administration, and both administrations take place after radiotherapy.

Methods for co-administration with an additional therapeutic agent are well known in the art.

Because of the adjuvant properties of the compounds of the present invention, their use may also combined with other therapeutic modalities including other vaccines, adjuvants, antigen, antibodies, and immune modulators.

In addition to the compounds of the present invention and compositions thereof described herein, the compositions or methods of the present invention may further comprise one or more additional substances which, because of their nature, can act to stimulate or otherwise utilize the immune system to respond to the cancer antigens present on the targeted tumor cell(s).

The compounds of the present invention can be used in combination with an immune checkpoint inhibitor, such as an immune checkpoint inhibitor selected from the group consisting of a CTLA-4 pathway antagonist, a PD-1 pathway antagonist, a Tim-3 pathway antagonist, a Vista pathway antagonist, a BTLA pathway antagonist, a LAG-3 pathway antagonist, or a TIGIT pathway antagonist.

The compounds of the present invention can be used in combination with an immuno-oncological agonist in combination with a T-cell receptor agonist, or in combination with a TNF receptor superfamily agonist or antagonist.

The compounds of the present invention can be used in combination with therapeutic antibodies or therapeutic nanobodies. In some embodiments, the mechanism of action of the therapeutic antibody is Antibody-Dependent Cell-Mediated Cytotoxicity (ADCC).

In additional embodiments of the methods described herein, the compounds of the present invention are used in combination with chemotherapeutic agents (e.g. small molecule pharmaceutical compounds) as known to the skilled person. Thus the methods further involve administering to the subject an effective amount of one or more chemotherapeutic agents as an additional treatment or a combination treatment.

Additional pharmacologically active substance(s) which can also be used together/in combination with the compound of formula (I)—or a pharmaceutically acceptable salt thereof—(including all individual embodiments or generic subsets of compounds (I)) or in the medical uses, uses, methods of treatment and/or prevention as herein (above and below) disclosed include, without being restricted thereto: hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide); aromatase inhibitors (e.g. anastrozole, letrozole, liarozole, vorozole, exemestane, atamestane); LHRH agonists and antagonists (e.g. goserelin acetate, luprolide); inhibitors of growth factors and/or of their corresponding receptors (growth factors are for example: platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4),) and/or their corresponding receptors; inhibitors are for example (anti-)growth factor antibodies, (anti-)growth factor receptor antibodies and tyrosine kinase inhibitors, such as for example afatinib, dacomitinib, canertinib, neratinib, avitinib, poziotinib, AV 412, PF-6274484, HKI 357, olmutinib, osimertinib, almonertinib, nazartinib, lazertinib, pelitinib, erlotinib, gefitinib, icotinib, sapitinib, lapatinib, varlitinib, vandetanib, TAK-285, AEE788, BMS599626/AC-480, GW 583340, necitumumab, panitumumab, cetuximab, amivantanab, pertuzumab, trastuzumab, trastuzumab emtansine, or inhibitors of mutant EGFR, an inhibitor of HER2 with exon 20 mutations, and hepatocyte growth factor (HGF, c-MET, e.g. emibetuzumab, amivantanab, savolitinib, cabozantinib, foretinib); antimetabolites (e.g. methotrexate, raltitrexed, 5-fluorouracil (5-FU), capecitabine, floxuridine, gemcitabine, mercaptopurine, thioguanine, cladribine, pentostatin, cytarabine (ara C), fludarabine, combination of trifluridine and tipiracil (=TAS102)); antitumor antibiotics (e.g. anthracyclins such as doxorubicin, doxil (pegylated liposomal doxorubicin hydrochloride), myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids e.g. vinblastine, vindesin, vinorelbin and vincristine; and taxanes such as paclitaxel, docetaxel, nab-paclitaxel (Abraxane)); angiogenesis inhibitors (e.g. tasquinimod, bevacizumab), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone); serine/threonine kinase inhibitors (e.g. PDK 1 inhibitors, Raf inhibitors, A-Raf inhibitors, B-Raf inhibitors, C-Raf inhibitors, mTOR inhibitors (e.g. rapamycin, temsirolimus, everolimus, ridaforolimus, zotarolimus, sapanisertib, Torin 1, dactosilib, GDC-0349, vs-5584; vistusertib; AZD8055), mTORC1/2 inhibitors, PI3K inhibitors, PI3Kα inhibitors (e.g. alpelisib, serabelisib, GDC-0077, HH-CYH33, AMG 511, buparlisib, dactolisib, pictilisib, taselisib), dual mTOR/PI3K inhibitors, STK 33 inhibitors, AKT inhibitors, PLK 1 inhibitors, inhibitors of CDK4/6 (e.g. palbociclib, ribociclib, abemaciclib, trilaciclib, PF-06873600), Aurora kinase inhibitors); tyrosine kinase inhibitors (e.g. PTK2/FAK inhibitors); protein protein interaction inhibitors (e.g. IAP inhibitors/SMAC mimetics, MCL-1 (e.g. AZD-5991, AMG-176, AMG-397, S64315, S63845, A-1210477), MDM2, MDM2/MDMX); MEK inhibitors (e.g. trametinib, cobimetinib, binimetinib, selumetinib, refametinib); SOS1-inhibitor (i.e. a compound that modulates/inhibits the GEF functionality of SOS1, e.g. by binding to SOS1 and preventing protein-protein interaction between SOS1 and a (mutant) Ras protein, e.g. KRAS; e.g. BAY-293), an inhibitor of GDP-loaded or GTP-loaded RAS and/or of any mutants thereof (i.e. a compound that modulates/inhibits the functionality of (mutant) RAS protein by, e.g., binding to GDP-loaded or GTP-loaded (mutant) RAS protein, e.g. KRAS, NRAS and/or HRAS, preferably KRAS); an irreversible inhibitor of KRAS G12C (AMG-510, MRTX849, ARS-324, GDC-6036); a reversible or irreversible binder to GDP-loaded (mutant) KRAS; a reversible or irreversible binderto GTP-loaded (mutant) KRAS; ALK inhibitors (e.g. crizotinib, alectinib, entrectinib, brigatinib, ceritinib); ERK inhibitors; FLT3 inhibitors; BRD4 inhibitors; IGF-1R inhibitors; TRAILR2 agonists; Bcl-xL inhibitors; Bcl-2 inhibitors (e.g. venetoclax, obatoclax, navitoclax, oblimersen); Bcl-2/Bcl-xL inhibitors; ErbB receptor inhibitors; BCR-ABL inhibitors; ABL inhibitors; Src inhibitors (e.g. dasatinib, ponatinib, bosutinib, vandetanib, KX-01, saracatinib, KX2-391, SU 6656, WH-4-023); rapamycin analogs (e.g. everolimus, temsirolimus, ridaforolimus, sirolimus); androgen synthesis inhibitors; androgen receptor inhibitors; DNMT inhibitors; HDAC inhibitors; ANG1/2 inhibitors; histone deacetylase inhibitor; an inhibitor of IL6; inhibitor ofJAK and/or any mutants thereof; an inhibitor of A-Raf and/or B-Raf and/or C-Raf and/or any mutants thereof (encorafenib, dabrafenib, vemurafenib, PLX-8394, RAF-709 (=example 131 in WO 2014/151616), LXH254, sorafenib, LY-3009120 (=example 1 in WO 2013/134243), lifirafenib, TAK-632, agerafenib, CCT196969, RO5126766, RAF265); an inhibitor of a receptor tyrosine kinase (RTK) and/or of any mutants thereof; an inhibitor of SHP2 and/or of any mutants thereof (e.g. SHP099, TNO155, RMC-4550, RMC-4630, IACS-13909); CYP17 inhibitors; radiopharmaceuticals; proteasome inhibitors (e.g. carfilzomib); immunotherapeutic agents such as immune checkpoint inhibitors (e.g. CTLA4, PD1, PD-L1, PD-L2, LAG3, SIRPalpha-antibodies, and TIM3 binding molecules/immunoglobulins (ipilimumab, nivolumab, pembrolizumab, tislelizumab, atezolizumab, avelumab, durvalumab, pidilizumab, PDR-001 (=spartalizumab), AMG-404, ezabenlimab, sintilimab, camrelizumab, toribalimab, tislelizumab,); ADCC (antibody-dependent cell-mediated cytotoxicity) enhancers (e.g. anti-CD33 antibodies, anti-CD37 antibodies, anti-CD20 antibodies); T-cell engagers, e.g. PSMA×CD3, B7H6/CD3 (as e.g. disclosed in WO2021/064137), DLL3/CD3 (as e.g. disclosed in WO2019/234220), e.g. bi-specific T-cell engagers (BiTEs®) like e.g. CD3×BCMA, CD3×CD33, CD3×CD19), cancer vaccines, MDM2-inhibitors, oncolytic viruses and various chemotherapeutic agents such as amifostin, anagrelid, clodronat, filgrastin, interferon, interferon alpha, leucovorin, procarbazine, levamisole, mesna, mitotane, pamidronate and porfimer. The compounds of the present invention can be used in combination with an OX40 agonist, an ICOS-ligand, a CD27 agonist, a GITR agonist, a Toll like receptor agonist.

In a preferred embodiment, additional pharmacologically active substance(s) which can also be used together/in combination with the compound of formula (I)—or a pharmaceutically acceptable salt thereof—(including all individual embodiments orgeneric subsets of compounds (I)) or in the medical uses, uses, methods of treatment and/or prevention as herein (above and below) disclosed include check-point inhibitors (ipilimumab, nivolumab, pembrolizumab, tislelizumab, atezolizumab, avelumab, durvalumab, pidilizumab, PDR-001 (=spartalizumab), AMG-404, ezabenlimab, sintilimab, camrelizumab, toribalimab, tislelizumab), taxanes (paclitaxel, docetaxel, nab-paclitaxel (Abraxane)), T-cell-engagers e.g. PSMA×CD3, B7H6/CD3 (as e.g. disclosed in WO2021/604137), DLL3/CD3 (as e.g. disclosed in WO2019/234220), e.g. bi-specific T-cell engagers (BiTEs®) like e.g. CD3×BCMA, CD3×CD33, CD3×CD19, cancer vaccines, MDM2-inhibitors, and oncolytic viruses.

In additional embodiments of the methods described herein, the compounds of the present invention are used in combination with chemotherapeutic agents and/or additional agents e.g. cancer-targeting therapies, for treating the indications as described in the methods herein. Thus the methods further involve administering to the subject an effective amount of one or more cancer-targeting agents as an additional treatment or a combination treatment.

In additional embodiments the methods described herein, the compounds of the present invention are used in combination with chemotherapeutic agents and/or additional agents for treating the indications as described in the methods herein and/or additional therapies such as radiotherapy and/or tumor excision.

In yet another aspect, the present invention relates a method for treating a disease or condition associated with or modulated by STING in a patient that includes the step of administering to a patient in need of such treatment a therapeutically effective amount of a compound of the present invention in combination with a therapeutically effective amount of one or more additional therapeutic agents described hereinbefore.

The use of the compound according to the invention in combination with the additional therapeutic agent may take place simultaneously or at staggered times.

The compound according to the invention and the one or more additional therapeutic agents may both be present together in one formulation or separately in two identical or different formulations, for example as a so-called kit-of-parts.

Thus, in a further aspect, the present invention provides a combination comprising a compound of general formula (I), and at least one further therapeutic agent.

A further aspect of the present invention is to provide a pharmaceutical composition comprising a compound of formula (I), or a pharmaceutically acceptable salt thereof, and at least one further therapeutic agent and one or more of pharmaceutically acceptable excipients.

In a further aspect, the invention provides a combination comprising a compound of formula (I), or a pharmaceutically acceptable salt thereof, and at least one further therapeutic agent for use in therapy.

In a further aspect, the invention provides a combination comprising a compound of formula (I), or a pharmaceutically acceptable salt thereof, and at least one further therapeutic agent for use in the treatment of a disease or condition in which modulation of STING is beneficial.

In a further aspect the invention provides a combination comprising a compound of formula (I), or a pharmaceutically acceptable salt thereof, and at least one further therapeutic agent for use in the treatment of cancer, e.g. canine or feline cancer.

In another aspect, this invention relates to a pharmaceutical composition which comprises a compound according to the invention and one or more additional therapeutic agent(s) described hereinbefore and hereinafter, optionally together with one or more inert carriers and/or diluents.

Other features and advantages of the present invention will become apparent from the following more detailed Examples which illustrate, by way of example, the principles of the invention.

Chemical Synthesis

List of Abbreviations

| | |
|---|---|
| aq | Aqueous |
| DCM | Dichloromethane |
| DIPEA | Diisopropylethylamine (Hünig's base) |
| DMF | Dimethylformamide |
| DMSO | Dimethylsulfoxide |
| PdCl₂dppf | [1,1′-bis-(diphenylphosphino)-ferrocenyl]-dichloro- |
| | palladium(II) |
| EtOH | Ethanol |
| EtOAc | Ethyl Acetate |
| h | Hours |
| HPLC | High Pressure Liquid Chromatography |
| K2CO3 | Potassium carbonate |
| LDA | Lithium Diisopropylamide |
| MeCN | Acetonitrile |
| MeOH | Methanol |
| MS | Mass spectrometry |
| NaHCO₃ | Sodium hydrogen carbonate (sodium bicarbonate) |
| NaOH | Sodium hydroxide |
| NFSI | N-Fluorobenzenesulfonimide |
| NMP | N-Methylpyrrolidone |
| NMR | Nuclear Magnetic Resonance |
| ppm | Part per million |
| RT | Room temperature |
| TFA | Trifluoroacetic acid |
| THF | Tetrahydrofuran |

Other features and advantages of the present invention will become apparent from the following more detailed examples which exemplarily illustrate the principles of the invention without restricting its scope.

General

Unless stated otherwise, all the reactions are carried out in commercially obtainable apparatuses using methods that are commonly used in chemical laboratories. Starting materials that are sensitive to air and/or moisture are stored under protective gas and corresponding reactions and manipulations there with are carried out under protective gas (nitrogen or argon). The compounds according to the invention are named in accordance with IUPAC guidelines. If a compound is to be represented both by a structural formula and by its nomenclature, in the event of a conflict the structural formula is decisive.

Chromatography

Thin layer chromatography is carried out on ready-made TLC plates of silica gel 60 on glass (with fluorescence indicator F-254) made by Merck.

A Biotage Isolera Four apparatus is used for automated preparative NP chromatography together with Interchim Puri Flash columns (50 μm, 12-300 g) or glass columns filled with silica gel made by Millipore (Granula Silica Si-60A 35-70 μm).

Preparative RP HPLC is carried out with columns made by Waters (Sunfire C18, 10 am, 30×100 mm Part. No. 186003971 or X-Bridge C18, 10 μm, 30×100 mm Part. No. 186003930). The compounds are eluted using either different gradients of H2O/acetonitrile or H2O/MeOH, where 0.1% TFA is added to the water, or with different gradients utilizing a basic aqueous buffersolution (1 Lwater contains 5 mL of an ammonium hydrogen carbonate solution (158 g per 1 L H2O) and 2 mL ammonia (7 mol/I solution in MeOH)) instead of the water-TFA-mixture.

The analytical HPLC (reaction monitoring) of intermediate compounds is carried out with columns made by Waters and Phenomenex. The analytical equipment is also provided with a mass detector in each case.

HPLC Mass Spectroscopy/UV Spectrometry

The retention times/MS-ESI+ for characterizing the example compounds according to the invention are determined using an HPLC-MS apparatus (high performance liquid chromatography with mass detector) e.g. made by Agilent. Compounds that elute at the injection peak are given the retention time tR=0.

Analytical HPLC Methods:

Acidic Method

| | |
|---|---|
| HPLC: | Agilent 1260 Infinity II |
| MS: | Agilent LC/MS (G6125B) |
| Column: | Sunfire C18 2.5 μm, 3.0 × 30 mm |
| Eluent: | A: 0.1% TFA (v/v) in H2O; B: MeCN (HPLC grade) |
| Detection: | MS: Positive and negative mode |
| Column temp.: | 60° C. |
| Gradient: | 0.00-0.20 min: 3% B (Flow 2.2 ml/min) |
| | 0.20-1.20 min: 3% to 100% B (Flow 2.2 ml/min) |
| | 1.20-1.25 min: 100% B (Flow 3.0 ml/min) |
| | 1.25-1.40 min: 100% B (Flow 3.0 ml/min) |

Basic Method

| | |
|---|---|
| HPLC: | Agilent 1260 Infinity II |
| MS: | Agilent LC/MS (G6125B) |
| Column: | X-Bridge C18, 2.5 μm, 3.0 × 30 mm |
| Eluant: | A: 0.1% NH4OH (v/v) in H2O; B: MeCN (HPLC grade) |
| Detection: | MS: Positive and negative mode |
| Column temp.: | 60° C. |
| Gradient: | 0.00-0.20 min: 3% B (Flow 2.2 ml/min) |
| | 0.20-1.20 min: 3% to 100% B (Flow 2.2 ml/min) |
| | 1.20-1.25 min: 100% B (Flow 3.0 ml/min) |
| | 1.25-1.40 min: 100% B (Flow 3.0 ml/min) |

Preparative HPLC Methods:

Acidic Method

| | |
|---|---|
| HPLC: | Agilent 1260 Infinity II |
| MS: | Agilent LC/MS (G6125B) |
| Column: | Sunfire C18 10 μm, 30 × 300 mm |
| Eluent: | A: 0.1% TFA (v/v) in H2O; B: MeCN (HPLC grade) |
| Detection: | MS: Positive and negative mode |
| Flow: | 50 ml/min |
| Column temp.: | 40° C. |

Basic Method

| | |
|---|---|
| HPLC: | Agilent 1260 Infinity II |
| MS: | Agilent LC/MS (G6125B) |
| Column: | X-Bridge C18, 10 μm, 30 × 300 mm |
| Eluant: | A: 0.1% NH4OH (v/v) in H2O; B: MeCN (HPLC grade) |
| Detection: | MS: Positive and negative mode |
| Flow: | 50 ml/min |
| Column temp.: | 40° C. |

Preparation of the Compounds According to the Invention

The compounds according to the present invention and their intermediates may be obtained using methods of synthesis which are known to the one skilled in the art and described in the literature of organic synthesis. These methods are intended as an illustration of the invention, without restricting its subject matter and the scope of the compounds claimed to these examples. Preferably, the compounds are obtained in analogous fashion to the methods of preparation explained more fully hereinafter, in particular as described in the experimental section. In some cases, the order in carrying out the reaction steps may be varied. Variants of the reaction methods that are known to the one skilled in the art but not described in detail here may also be used.

The general processes for preparing the compounds according to the invention will become apparent to the one skilled in the art studying the following schemes. Starting materials may be prepared by methods that are described in the literature or herein, or may be prepared in an analogous or similar manner. Any functional groups in the starting materials or intermediates may be protected using conventional protecting groups. These protecting groups may be cleaved again at a suitable stage within the reaction sequence using methods familiar to the one skilled in the art.

One method for the preparation of compounds of formula (I) is exemplified in Scheme I: Indazoles B can be synthesized from ortho-methyl aniline derivatives A. Subsequent iodination leads to 3-iodo-indazoles C. Intermediates D can be obtained e.g. by Chan-Lam coupling utilizing (6-fluoropyridin-3-yl)boronic acid. Conversion into intermediates F can be achieved e.g. via Suzuki coupling with intermediates E. Finally, compounds of formula (I) are synthesized e.g. by nucleophilic aromatic substitution. The products are isolated by conventional means and preferably purified by chromatography.

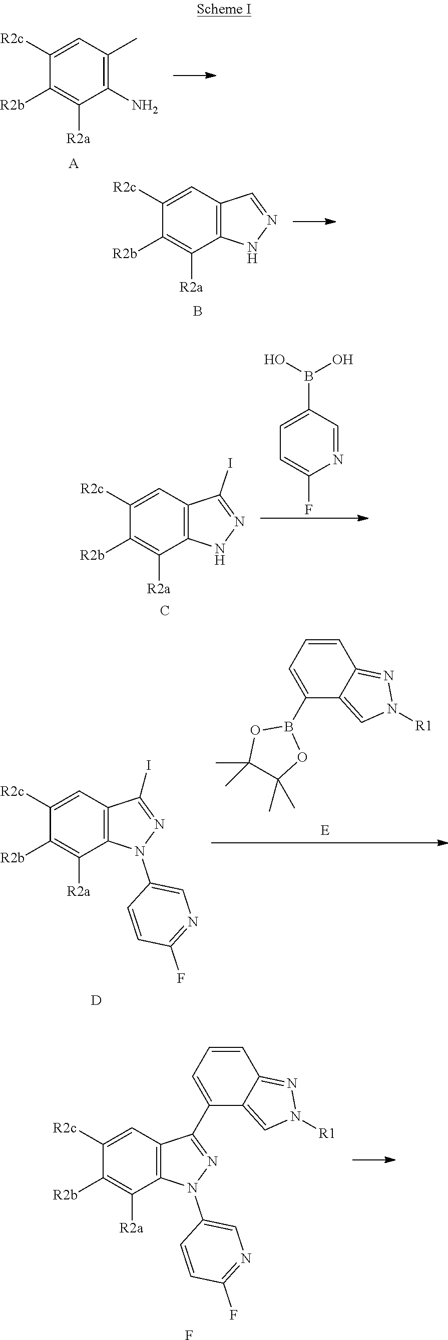

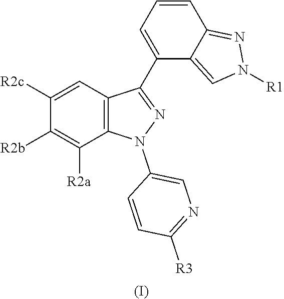

### PREPARATION OF INTERMEDIATES

### Intermediate 1

### 1-(6-Fluoropyridin-3-yl)-3-iodo-7-methyl-1H-indazole

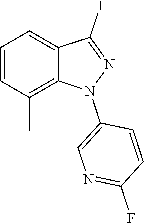 [SMILES: Cc1cccc2c(I)nn(-c3ccc(F)nc3)c12]

To a stirred reaction of 3-iodo-7-methyl-1H-indazole (16 g; 62 mmol in DCM (200 ml) was added copper acetate (16,889 g; 93 mmol), pyridine (9,796 g; 124 mmol), and 6-fluoropyridine-3-boronic acid (14,851 g; 105 mmol). The reaction mixture was then stirred at RT for 72 h. The reaction was filtered through celite, the filtrate was concentrated, and the crude was purified using silica chromatography (EtOAc:Hexane) to give the title compound (13 g).

1H NMR (400 MHz, DMSO-d6) δ ppm 2.09 (s, 3H), 7.25 (m, 1H), 7.34 (m, 1H), 7.42 (m, 2H), 8.28 (ddd, J=8.65, 7.07, 2.79 Hz, 1H), 8.53 (d, J=2.15 Hz, 1H).

### Intermediate 2

### 1-(6-Fluoropyridin-3-yl)-2′,7-dimethyl-1H,2′H-3,4′-biindazole

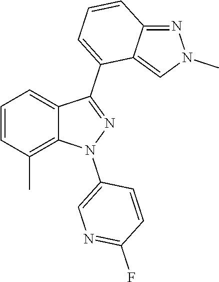 [SMILES: Cc1cccc2c(-c3cccc4nn(C)cc34)nn(-c3ccc(F)nc3)c12]

A mixture of 1-(6-fluoropyridin-3-yl)-3-iodo-7-methyl-1H-indazole (Intermediate 1, 1.0 g, 2.8 mmol), 2-methyl-4-(tetramethyl-1,3,2-dioxaborolan-2-yl)-2H-indazole (1.1 g, 4.2 mmol), 1,1′-bis-(diphenylphosphino)ferrocene]dichloropalladium(II) (0.21 g, 0.28 mmol), and Na₂CO₃ (0.9 g, 8.5 mmol) was stirred in dioxane (20 ml) and water (5 ml) under argon atmosphere for 2 h at 110° C. At RT, water (100 ml) was added, and the mixture was extracted with EtOAc. The combined organic layers were dried over MgSO4, concentrated in vacuo and the product purified by RP HPLC and gave the title compound (0.8 g).

### EXAMPLES

### Example 1

### 2-[(3R)-1-(5-{2″,7-dimethyl-1H,2″H-[3,4″-biindazol]-1-yl}pyridin-2-yl)pyrrolidin-3-yl]acetic acid

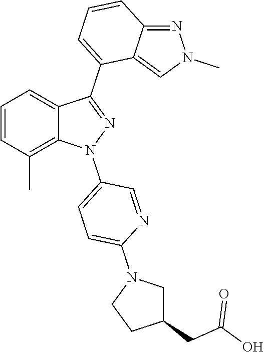 [SMILES: Cc1cccc2c(-c3cccc4nn(C)cc34)nn(-c3ccc(N4CC[C@H](CC(=O)O)C4)nc3)c12]

To a solution of Intermediate 2 (80 mg, 0.22 mmol) in anhydrous NMP (2 ml) was added methyl (R)-pyrrolidine-3-acetate HCl (106 mg, 0.56 mmol) followed by DIPEA (145 mg, 1.12 mmol) and the mixture was heated at 140° C. for 7 h. The reaction was allowed to cool and NaOH(aq) (4M, 300 μl, 1.2 mmol) was added and it was stirred at RT for 1 h. The reaction mixture was acidified with TFA and was purified by preparative HPLC (acidic method) to give the title compound (87 mg).

1H NMR (400 MHz, DMSO-d6) δ ppm 1.76 (dq, J=12.29, 8.45 Hz, 1H), 2.21 (s, 3H), 2.34 (m, 1H), 2.48 (m, 2H), 2.67 (m, 1H), 3.20 (dd, J=10.65, 7.73 Hz, 1H), 3.51 (m, 1H), 3.66 (m, 1H), 3.81 (dd, J=10.58, 7.29 Hz, 1H), 4.06 (br d, J=31.05 Hz, 1H), 4.20 (s, 3H), 6.78 (br d, J=9.12 Hz, 1H), 7.25 (m, 2H), 7.42 (dd, J=8.62, 6.97 Hz, 1H), 7.67 (d, J=8.62 Hz, 1H), 7.77 (d, J=6.72 Hz, 1H), 7.94 (dd, J=9.13, 2.28 Hz, 1H), 8.09 (d, J=7.10 Hz, 1H), 8.41 (d, J=2.53 Hz, 1H), 8.61 (s, 1H)

HPLC (Acidic): Rt=0.802 min ((M+H)+481.2)

### Example 2

### 2-[(3S)-1-(5-{2″,7-dimethyl-1H,2″H-[3,4″-biindazol]-1-yl}pyridin-2-yl)pyrrolidin-3-yl]acetic acid

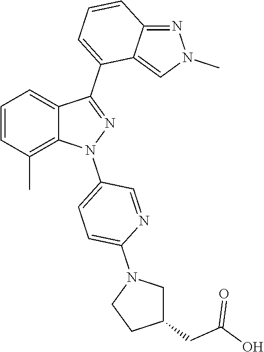 [SMILES: Cc1cccc2c(-c3cccc4nn(C)cc34)nn(-c3ccc(N4CC[C@@H](CC(=O)O)C4)nc3)c12]

Using the method described for Example 1: Intermediate 2 (50 mg) with methyl (S)-pyrrolidine-3-acetate HCl (65 mg) gave the title compound (19.6 mg).

1H NMR (400 MHz, DMSO-d6) δ ppm 1.75 (dq, J=12.32, 8.40 Hz, 1H), 2.20 (s, 4H), 2.47 (d, J=7.35 Hz, 2H), 2.65 (dquin, J=14.93, 7.36, 7.36, 7.36, 7.36 Hz, 1H), 3.17 (dd, J=10.58, 7.67 Hz, 1H), 3.48 (m, 1H), 3.63 (m, 1H), 3.79 (dd, J=10.52, 7.35 Hz, 1H), 4.20 (s, 3H), 6.64 (d, J=9.00 Hz, 1H), 7.24 (m, 1H), 7.42 (dd, J=8.62, 6.97 Hz, 1H), 7.66 (d, J=8.62 Hz, 1H), 7.77 (d, J=6.59 Hz, 1H), 7.82 (dd, J=8.93, 2.60 Hz, 1H), 8.09 (dd, J=7.60, 1.01 Hz, 1H), 8.37 (d, J=2.53 Hz, 1H), 8.61 (s, 1H)

HPLC (Acidic): Rt=0.865 min ((M+H)+467)

### Example 3

### 2-[4-(5-{2′,7-dimethyl-1H,2′H-[3,4′-biindazol]-1-yl}pyridin-2-yl)morpholin-2-yl]acetic acid

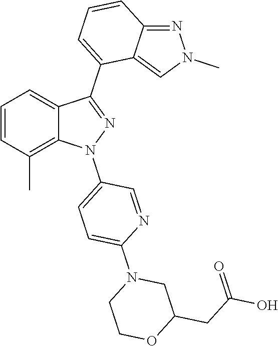 [SMILES: Cc1cccc2c(-c3cccc4nn(C)cc34)nn(-c3ccc(N4CCOC(CC(=O)O)C4)nc3)c12]

Using the method described for Example 1: Intermediate 2 (50 mg) with methyl 2-(morpholin-2-yl)acetate HCl (72 mg) after preparative HPLC gave the title compound (57.6 mg).

HPLC (Acidic): Rt=0.947 min ((M+H)+483.2)

### Example 4

### 2-(5-{2″,7-dimethyl-1H,2″H-[3,4″-biindazol]-1-yl}pyridin-2-yl)-2-azabicyclo[2.2.1]heptane-5-carboxylic acid

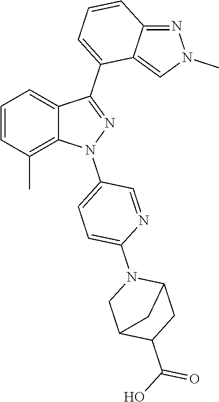 [SMILES: Cc1cccc2c(-c3cccc4nn(C)cc34)nn(-c3ccc(N4CC5CC4CC5C(=O)O)nc3)c12]

Using the method described for Example 1: Intermediate 2 (50 mg) with methyl 2-azabicyclo[2.2.1]heptane-5-carboxylate (57 mg) after preparative HPLC gave the title compound (69 mg).

HPLC (Acidic): Rt=0.811 min ((M+H)+479.2)

### Example 5

### (1S,4R,5R)-2-(5-{2″,7-dimethyl-1H,2″H-[3,4″-biindazol]-1-yl}pyridin-2-yl)-2-azabicyclo[2.2.1]heptane-5-carboxylic acid and 1R,4S,5S)-2-(5-{2′,7-dimethyl-1H,2′H-[3,4′-biindazol]-1-yl}pyridin-2-yl)-2-azabicyclo[2.2.1]heptane-5-carboxylic acid

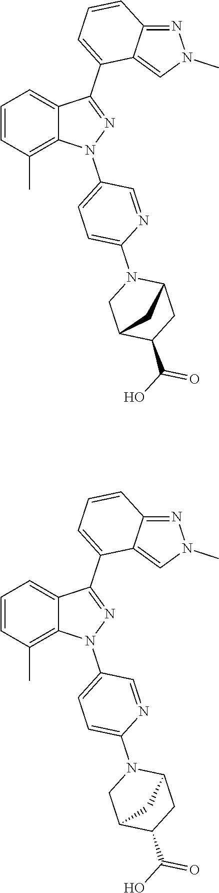 [SMILES: Cc1cccc2c(-c3cccc4nn(C)cc34)nn(-c3ccc(N4C[C@@H]5C[C@H]4C[C@H]5C(=O)O)nc3)c12 ; Cc1cccc2c(-c3cccc4nn(C)cc34)nn(-c3ccc(N4C[C@H]5C[C@@H]4C[C@@H]5C(=O)O)nc3)c12]

Using the method described for Example 1: Intermediate 2 (50 mg) with methyl (1S,4R,5R)-2-azabicyclo[2.2.1]heptane-5-carboxylate (57 mg) gave the title compound (56 mg).

1H NMR (400 MHz, DMSO-d6) δ ppm 1.68 (m, 1H), 1.76 (m, 1H), 1.99 (m, 3H), 2.22 (s, 5H), 2.66 (m, 1H), 2.93 (br s, 2H), 3.25 (br d, J=9.63 Hz, 2H), 3.52 (dd, J=9.82, 3.49 Hz, 1H), 4.20 (s, 3H), 4.70 (br s, 1H), 6.79 (br d, J=7.10 Hz, 2H), 7.25 (m, 3H), 7.42 (dd, J=8.62, 6.97 Hz, 2H), 7.66 (d, J=8.74 Hz, 2H), 7.76 (d, J=6.59 Hz, 2H), 7.91 (dd, J=8.93, 2.09 Hz, 2H), 8.08 (d, J=7.10 Hz, 2H), 8.39 (d, J=2.53 Hz, 2H), 8.61 (s, 2H)

HPLC (Acidic): Rt=0.811 min ((M+H)+479.2)

### Example 6

### 2-(5-{2′,7-dimethyl-1H,2′H-[3,4′-biindazol]-1-yl}pyridin-2-yl)-2-azabicyclo[2.2.2]octane-5-carboxylic acid

(Mixture of stereoisomers)

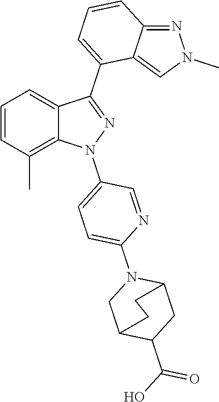 [SMILES: Cc1cccc2c(-c3cccc4nn(C)cc34)nn(-c3ccc(N4CC5CCC4CC5C(=O)O)nc3)c12]

Using the method described for Example 1: Intermediate 2 (50 mg) with 2-azabicyclo[2.2.2]octane-5-carboxylic acid (0.35 mmol) after preparative HPLC gave the title compound (42 mg).

HPLC (Acidic): Rt=0.839 min ((M+H)+493.2)

### Examples 7-10

Example 6 was submitted to chiral preparative HPLC separation using the conditions described below to yield the following stereoisomers:
(1S,4R,5R)-2-(5-{2′,7-dimethyl-1H,2′H-[3,4′-biindazol]-1-yl}pyridin-2-yl)-2-azabicyclo[2.2.2]octane-5-carboxylic acid (single stereoisomer)
(1R,4S,5S)-2-(5-{2′,7-dimethyl-1H,2′H-[3,4′-biindazol]-1-yl}pyridin-2-yl)-2-azabicyclo[2.2.2]octane-5-carboxylic acid (single stereoisomer)
(1S,4R,5S)-2-(5-{2′,7-dimethyl-1H,2′H-[3,4′-biindazol]-1-yl}pyridin-2-yl)-2-azabicyclo[2.2.2]octane-5-carboxylic acid (single stereoisomer)
(1R,4S,5R)-2-(5-{2′,7-dimethyl-1H,2′H-[3,4′-biindazol]-1-yl}pyridin-2-yl)-2-azabicyclo[2.2.2]octane-5-carboxylic acid (single stereoisomer)

Chiral Preparative HPLC

| | |
|---|---|
| HPLC: | Sepiatec PrepSFC50 |
| Column: | CHIRAL ART ® Cellulose-SJ_10 × 250 mm_5 μm |
| Eluent: | A: 80% scCO2; B: 20% MeOH + 20 mM NH₃ |
| Detection: | UV: 220 nm |
| Column temp.: | 40° C. |
| Flow: | 15 ml/min |
| Gradient: | isocratic |

### Example 11

### 1-[(1R,4R)-5-(5-{2″,7-dimethyl-1H,2″H-[3,4″-biindazol]-1-yl}pyridin-2-yl)-2,5-diazabicyclo[2.2.1]heptan-2-yl]ethan-1-one (single stereoisomer)

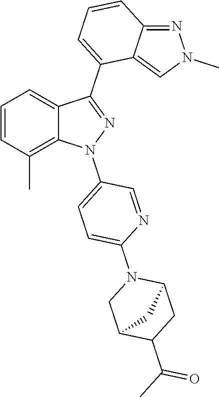 [SMILES: CC(=O)C1C[C@H]2C[C@@H]1CN2c1ccc(-n2nc(-c3cccc4nn(C)cc34)c3cccc(C)c32)cn1]

A mixture of Intermediate 2 (50 mg, 0.14 mmol), (1R,4R)-2,5-diazabicyclo[2.2.1]heptane (30 mg, 0.31 mmol), and DIPEA (90.4 mg, 0.7 mmol) in anhydrous NMP (2 ml) was heated for 15 h at 140° C. The mixture was allowed to cool to RT and acetic anhydride (57 mg, 0.56 mmol) and DIPEA (90 mg, 0.7 mmol) were added, and it was stirred at RT for 1 h. The product was purified by preparative HPLC (acidic) to give the title compound (20.5 mg).

HPLC (Acidic): Rt=0.859 min ((M+H)+478.2)

### Example 12

### 1-[(1S,4S)-5-(5-{2″,7-dimethyl-1H,2″H-[3,4″-biindazol]-1-yl}pyridin-2-yl)-2,5-diazabicyclo[2.2.1]heptan-2-yl]ethan-1-one (single stereoisomer)

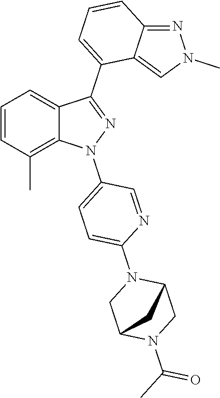 [SMILES: CC(=O)N1C[C@@H]2C[C@H]1CN2c1ccc(-n2nc(-c3cccc4nn(C)cc34)c3cccc(C)c32)cn1]

Using the method described for Example 11: Intermediate 2 with (1S,4S)-2,5-diazabicyclo[2.2.1]heptane after preparative HPLC gave the title compound.

### Example 13

### 1-[5-(5-{2′,7-dimethyl-1H,2′H-[3,4′-biindazol]-1-yl}pyridin-2-yl)-2,5-diazabicyclo[2.2.2]octan-2-yl]ethan-1-one (mixture of stereoisomers)

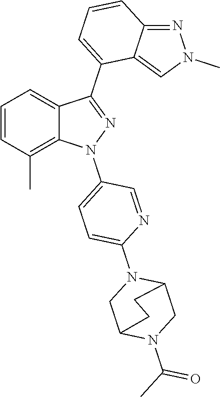 [SMILES: CC(=O)N1CC2CCC1CN2c1ccc(-n2nc(-c3cccc4nn(C)cc34)c3cccc(C)c32)cn1]

Using the method described for Example 11: Intermediate 2 (50 mg) with 2,5-diazabicyclo[2.2.2]octane dihydrochloride (68 mg) after preparative HPLC gave the title compound (23 mg).

HPLC (Acidic): Rt=0.896 min ((M+H)+492.2)

### Examples 14-15

Example 13 was submitted to chiral preparative HPLC separation using the conditions described below to yield the following stereoisomers:
1-[(1R,4R)-5-(5-{2′,7-dimethyl-1H,2′H-[3,4′-biindazol]-1-yl}pyridin-2-yl)-2,5-diazabicyclo[2.2.2]octan-2-yl]ethan-1-one (single stereoisomer)1-[(1S,4S)-5-(5-{2′,7-dimethyl-1H,2′H-[3,4′-biindazol]-1-yl}pyridin-2-yl)-2,5-diazabicyclo[2.2.2]octan-2-yl]ethan-1-one (single stereoisomer)

Chiral Preparative HPLC

| | |
|---|---|
| HPLC: | Sepiatec PrepSFC50 |
| Column: | CHIRAL ART ® Amylose_10 × 250 mm_5 μm |
| Eluent: | A: 60% scCO2; B: 40% MeOH + 20 mM NH₃ |
| Detection: | UV: 220 nm |
| Column temp.: | 40° C. |
| Flow: | 15 ml/min |
| Gradient: | isocratic |

### Example 14

### 1-[(1R,4R)-5-(5-{2′,7-dimethyl-1H,2′H-[3,4′-biindazol]-1-yl}pyridin-2-yl)-2,5-diazabicyclo[2.2.2]octan-2-yl]ethan-1-one (single stereoisomer)

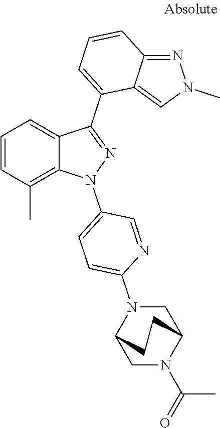 [SMILES: CC(=O)N1C[C@H]2CC[C@@H]1CN2c1ccc(-n2nc(-c3cccc4nn(C)cc34)c3cccc(C)c32)cn1]

### Example 15

### 1-[(1S,4S)-5-(5-{2′,7-dimethyl-1H,2′H-[3,4′-biindazol]-1-yl}pyridin-2-yl)-2,5-diazabicyclo[2.2.2]octan-2-yl]ethan-1-one (single stereoisomer)

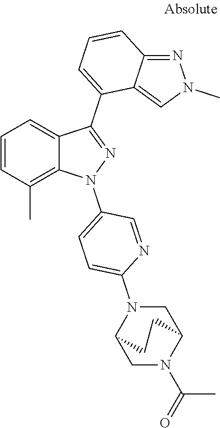 [SMILES: CC(=O)N1C[C@@H]2CC[C@H]1CN2c1ccc(-n2nc(-c3cccc4nn(C)cc34)c3cccc(C)c32)cn1]

### Example 16

### 2-(5-{2′,7-dimethyl-1H,2′H-[3,4′-biindazol]-1-yl}pyridin-2-yl)-2-azabicyclo[2.2.1]heptane-5-carboxylic acid (mixture of stereoisomers)

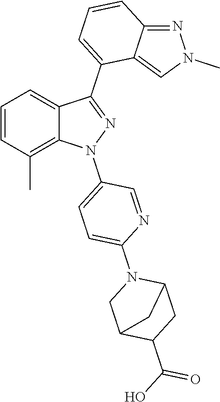 [SMILES: Cc1cccc2c(-c3cccc4nn(C)cc34)nn(-c3ccc(N4CC5CC4CC5C(=O)O)nc3)c12]

Using the method described for Example 1: Intermediate 2 (50 mg) with methyl 2-azabicyclo[2.2.1]heptane-5-carboxylate (57.1 mg, 0.35 mmol) gave the title compound (55 mg) after preparative HPLC (acidic).

HPLC (Acidic): Rt=0.846 min ((M+H)+479.0)

### Example 17

### (1S,4R,5R)-2-(5-{2′,7-dimethyl-1H,2′H-[3,4′-biindazol]-1-yl}pyridin-2-yl)-2-azabicyclo[2.2.1]heptane-5-carboxylic acid and (1R,4S,5S)-2-(5-{2′,7-dimethyl-1H,2′H-[3,4′-biindazol]-1-yl}pyridin-2-yl)-2-azabicyclo[2.2.1]heptane-5-carboxylic acid (mixture of stereoisomers)

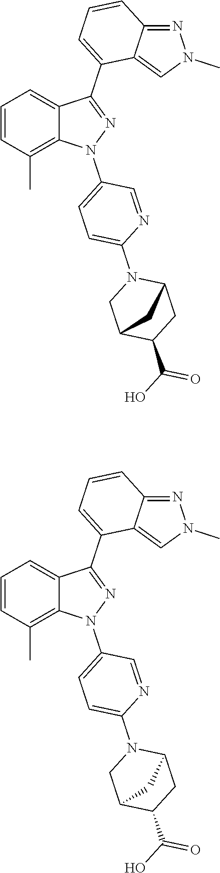 [SMILES: Cc1cccc2c(-c3cccc4nn(C)cc34)nn(-c3ccc(N4C[C@@H]5C[C@H]4C[C@H]5C(=O)O)nc3)c12 ; Cc1cccc2c(-c3cccc4nn(C)cc34)nn(-c3ccc(N4C[C@H]5C[C@@H]4C[C@@H]5C(=O)O)nc3)c12]

Using the method described for Example 1: Intermediate 2 (200 mg) with cis-methyl 2-azabicyclo[2.2.1]heptane-5-carboxylate (215 mg, 1.12 mmol) gave the title compound (55 mg) after preparative HPLC (acidic).

HPLC (Acidic): Rt=0.859 min ((M+H)+479.2)

### Examples 18-19

Example 16 was submitted to chiral preparative HPLC separation using the conditions described below to yield the following stereoisomers:
(1R,4S,5S)-2-(5-{2′,7-dimethyl-1H,2′H-[3,4′-biindazol]-1-yl}pyridin-2-yl)-2-azabicyclo[2.2.1]heptane-5-carboxylic acid (single stereoisomer)
(1S,4R,5R)-2-(5-{2′,7-dimethyl-1H,2′H-[3,4′-biindazol]-1-yl}pyridin-2-yl)-2-azabicyclo[2.2.1]heptane-5-carboxylic acid (single stereoisomer)

Chiral Preparative HPLC

| | |
|---|---|
| HPLC: | Sepiatec PrepSFC100 |
| Column: | CHIRAL ART ® Amylose-C_neo_20 × 250 nm_5 μm |
| Eluent: | A: 60% scCO2; B: 40% MeOH + 20 mM NH₃ |
| Detection: | UV: 220 nm |
| Column temp.: | 40° C. |
| Flow: | 60 ml/min |
| Gradient: | isocratic |

### Example 18

### (1R,4S,5S)-2-(5-{2′,7-dimethyl-1H,2′H-[3,4′-biindazol]-1-yl}pyridin-2-yl)-2-azabicyclo[2.2.1]heptane-5-carboxylic acid (single stereoisomer)

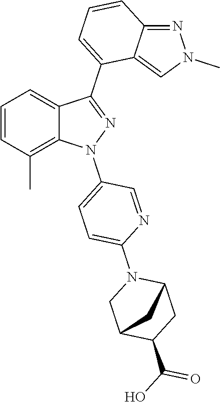 [SMILES: Cc1cccc2c(-c3cccc4nn(C)cc34)nn(-c3ccc(N4C[C@@H]5C[C@H]4C[C@H]5C(=O)O)nc3)c12]

¹H NMR (400 MHz, DMSO-d₆) δ ppm 1.72 (m, 2H), 1.99 (m, 2H), 2.22 (s, 3H), 2.66 (dd, J=8.49, 5.45 Hz, 1H), 2.94 (br s, 1H), 3.26 (br d, J=9.76 Hz, 1H), 3.53 (dd, J=9.76, 3.42 Hz, 1H), 4.20 (s, 3H), 4.71 (br s, 1H), 6.82 (br d, J=8.24 Hz, 1H), 7.25 (m, 2H), 7.42 (dd, J=8.62, 6.97 Hz, 1H), 7.67 (d, J=8.62 Hz, 1H), 7.76 (d, J=6.84 Hz, 1H), 7.93 (dd, J=9.12, 2.15 Hz, 1H), 8.09 (d, J=7.35 Hz, 1H), 8.40 (d, J=2.53 Hz, 1H), 8.61 (s, 1H).

### Example 19

### (1S,4R,5R)-2-(5-{2′,7-dimethyl-1H,2′H-[3,4′-biindazol]-1-yl}pyridin-2-yl)-2-azabicyclo[2.2.1]heptane-5-carboxylic acid (single stereoisomer)

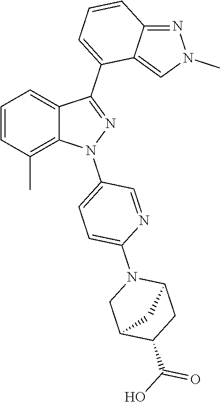 [SMILES: Cc1cccc2c(-c3cccc4nn(C)cc34)nn(-c3ccc(N4C[C@H]5C[C@@H]4C[C@@H]5C(=O)O)nc3)c12]

¹H NMR (400 MHz, DMSO-d₆) δ ppm 1.72 (m, 2H), 1.99 (m, 2H), 2.23 (s, 3H), 2.66 (dd, J=8.43, 5.64 Hz, 1H), 2.94 (br s, 1H), 3.26 (br d, J=9.63 Hz, 1H), 3.53 (dd, J=9.76, 3.42 Hz, 1H), 4.20 (s, 3H), 4.71 (br s, 1H), 6.82 (br d, J=7.10 Hz, 1H), 7.25 (m, 2H), 7.42 (dd, J=8.62, 6.97 Hz, 1H), 7.67 (d, J=8.62 Hz, 1H), 7.76 (d, J=6.72 Hz, 1H), 7.93 (dd, J=9.06, 2.22 Hz, 1H), 8.09 (d, J=7.35 Hz, 1H), 8.40 (d, J=2.53 Hz, 1H), 8.61 (s, 1H).

Pharmacological Activity

Biological Examples

The compounds of the present disclosure were tested in two assays as described below. That is, the compounds were tested in a dog differential scanning fluorimetry assay, as well as a dog whole blood assay. Representative results from the compounds of the present invention are compiled in Tables 1 and 2 below. Further assays for testing the compounds are also described in the following.

Dog Differential Scanning Fluorimetry (DSF) Assay

Dog STING (dSTING) Protein Production and Purification

The protein used for the biophysical experiments was a recombinant dog STING protein comprising its cytosolic cGAMP binding ectodomain. A codon optimized DNA sequence (for expression in Escherichia coli) encoding amino acid residues 149 to 375 of dog STING was synthesized by GeneArt (Regensburg, Germany) and inserted into a pET17b E. coli expression vector. The protein construct encodes an N-terminal 8× His-tag followed by tobacco etch virus protease (TEV) cleavage site and the above STING gene sequence. The resulting protein sequence for the used dog STING protein (SEQ ID No. 1) is listed below:

His-TEV-dSTING
MHHHHHHHHENLYFQSGEKRNFNVAHGLAWSYFIGYLRLILPGLPARIQ
ALHNNMLQGIGSHRLHILFPLDCGVPDDLSVVDPNIRFLYELPQQSANR
AGIKRRVYTNSVYELLEKGQPAGICVLEYATPLQTLFAMSQDGRAGFSR
EDRLEQAKLFCRTLEDILADAPELQNNCRLIVYQEPAEGSSFSLSQEIL
RHLRQEEREVTMGSMDTSIVPTSSTLSQEPNLFISGLEQPLPLRTDIF

For expression of the above recombinant dog STING, the construct was transformed into E. coli BL21 DE3 strain and grown in shake flasks in LB-medium at 15° C. Expression was induced by addition of isopropyl β-D-1-thiogalactopyranoside to a final concentration of 1 mM and cultures shaken overnight. Cell pellets were centrifuged and stored at −70° C. until further use. Protein was purified by cell thawing in lysis buffer (20 mM TRIS-HCl, pH 8, 500 mM NaCl, 1 mM DTT, 0.5 mg/ml lysozyme, Complete Protease Inhibitor (Roche) and DNase (Roche)), followed by metal affinity purification using Ni-NTA resins and elution buffer consisting of 20 mM TRIS-HCl, pH 8, 500 mM NaCl, 1 mM DTT, 300 mM imidazole. Cleavage of the His-Tag using TEV-protease took place during dialysis in size exclusion buffer (20 mM TRIS-HCl, pH 8, 100 mM NaCl, 1 mM DTT) over night. To further purify the target protein a reverse Nickel affinity column was used and the flow through was applied to a size exclusion chromatography. The peak fraction was collected and concentrated to 5 mg/mL.

Biophysical Assay—Determination of the Increase of Stability of Dog STING Protein Against Thermal Denaturation, Differential Scanning Fluorimetry (DSF)

The binding affinity of the compounds of the invention was demonstrated using a thermal shift assay that measures the stability of a suitable protein material of dog STING against thermal denaturation in the presence of compounds. In this assay, the unfolding temperature of a protein is monitored in the presence of a fluorescent dye which exhibits affinity for the hydrophobic amino acids of the protein that are buried in its folded state and are gradually exposed during unfolding. Dye fluorescence is quenched in aqueous environment and increases upon association of the dye with the hydrophobic parts of the unfolding protein. A plot of the fluorescence intensity as a function of temperature typically displays a sigmoidal curve that is interpreted by a two-state model of protein unfolding (Differential Scanning Fluorimetry). The inflection point of the curve represents the “melting” temperature of the protein (Tm) which is calculated numerically using the Boltzmann equation.

The thermal stability of the dog STING protein was measured in assay buffer containing 20 mM Tris, 150 mM NaCl at pH7.5. The assay uses 384-Well qPCR Plates (Catalog #781358, BRAND), Microseal®‘B’ Adhesive Seals for PCR Plates (Catalog #MSB-1001, BIO-RAD) and was run on a CFX384 Real-Time System (Bio-Rad). A DMSO stock solution of SYPRO orange (SIGMA 55692-500UL) was prepared. Compound stock solutions (10 mM in DMSO) were diluted 1:2 in DMSO to an intermediate compound concentration of 5 mM and then further diluted 1:40 in assay buffer resulting in a compound concentration of 125 μM and 2.5% DMSO. Fluorescent dye stock solution (5000× SYPRO Orange) was then mixed with target protein and buffer to a concentration of 15 μM Protein and 25× SYPRO Orange. 2 μl of this protein-dye-mixture was added to 8 μl compound solution. Final volume was 10 μL. 3-6 well positions were used as negative control (protein with 2% DMSO). The plates were prepared for duplicate measurement and centrifuged for 2 min at 1000 g. In the measurement, 160 cycles of 0.5° C. were used (temperature ramp 15 s/cycle, 15° C. to 95° C.).

Final Assay concentrations for compound characterization were 100 μM compound, 3 μM target protein, 5× SYPRO Orange, and 2% DMSO in 10 μl. All dispensing steps were performed using a HamiltonStar pipetting robot (Hamilton).

Dissociation curves were processed in Bio-Rad CFX Manager. Peak type was set to “negative”. Compound codes for screen were assigned in the plate layout.

Two replicates of TM measurements were averaged, and the standard deviation was calculated. In cases of SD>1.5° C. the measurement was repeated.

The melting point (Tm) obtained for STING protein alone was subtracted from T obtained for protein incubated with ligand to generate ΔTm values.

Dog Whole Blood Assay

For the detection of STING activation in physiological environment dog whole blood (dWB) was stimulated by the cyclic dinucleotide cGAMP or a test compound. Pathway activity was monitored by measuring the IFNb production.

Compounds were delivered as 10 mM DMSO solution, diluted and transferred by using an Echo acoustic dispenser to the 384well assay plate (Greiner #781182), pre-filled with 10 μl 1× HBSS in each well (10× HBSS (+Ca/+Mg), #14065-049, Gibco). Typically, 8 concentrations were used with the highest concentration at 10 μM in the final assay volume followed by approximately 1:4 dilution steps. DMSO concentration was set to 0.1% in the final assay volume. The 384-well assay plate contained 20 test compounds and DMSO in control and cGAMP standard wells. The dog whole blood was collected as Na-Citrate blood (e.g., 3.8% in Monovettes from Sarstedt) and kept at 4° C. overnight until use in the assay. 80 μl of the whole blood samples were transferred to each well of the 384-well assay plates filled with compound/1×HBSS. Blood plates were kept at room temperature for 60 minutes and continuous shaking with 450 rpm, covered with the lid, but not sealed. A 10× cGAMP assay solution was diluted from a 2 mM stock solution in 1×HBSS immediately before use at room temperature. 10 μl of the 10× cGAMP/HBSS were added to the high control wells, whereas HBSS only was added to all compound and low control wells. After covering assay plates with the lid, blood plates were kept for a 4 h incubation at 37° C. in the incubator, without shaking. For the detection of IFNb in dog plasma the ELISA Kit for Canine Interferon Beta (Biotrend #SEA222Ca) was used. Whole blood assay plates were centrifuged at 1000 g for 10 minutes at 8° C. 40 μl of supernatant was transferred with a 96 well pipetting robotics from the 384 well whole blood plate to the corresponding 96 well ELISA plate, pre-filled with 601 assay diluent in each well. Plates were sealed with microplate seals and kept at 4° C. overnight again. ELISA plates were brought to room temperature, followed by a 1 h incubation at 37° C. in the incubator. Detection Reagent A working solution was prepared by diluting Detection Reagent A 1:100 in Assay Reagent A. Liquid was then removed from the 96 well ELISA plate to add 100 μL Detection Reagent A working solution to each well. ELISA plates were covered with the plate sealer and incubated for 1 hour at 37° C. in the incubator. The 1× Wash buffer was prepared by diluting the 30× Wash Buffer Concentrate in H2O. Detection Reagent B working solution was prepared by diluting Detection Reagent B 1:100 in Assay Reagent B. The ELISA plates were washed three times with 350 μl wash buffer and afterwards inverted and blotted against absorbent paper to remove any liquid. 100 μL of Detection Reagent B working solution was added to each well of the ELISA plates, which are then covered with the plate sealer and incubated for 30 minutes at 37° C. in the incubator. After incubation the ELISA assay plates were washed five times with 350 μl wash buffer and were again inverted and blotted against absorbent paper to remove any remaining liquid. 90 μL of TMB Substrate was added to each well of the ELISA plates, which were then covered with the plate sealer and incubated for 15 minutes at 37° C. in the incubator. The reaction was stopped by adding 50 μL stop solution and absorbance was measured at 450 nm immediately.

Data Evaluation and Calculation:

For data evaluation and calculation, % control calculation of each well was based on the mean of high (cGAMP stimulated control) and mean of low (unstimulated control) controls by using the following standard 4 parameter logistic regression formula:

[y=(a−d)/(1+(x/c)\hat{ }b)+d]
a=low value,
d=high value,
x=conc M,
c=EC50 M,
b=slope.

Dog Liver Microsome (dLM) Assay

The metabolic degradation of the test compound is assayed at 37° C. with pooled liver microsomes from dogs (Beagle). The final incubation volume of 100 μl per time point contains TRIS buffer pH 7.6 at RT (0.1 M), magnesium chloride (5 mM), microsomal protein (1 mg/ml) and the test compound at a final concentration of 1 μM. Following a short preincubation period at 37° C., the reactions were initiated by addition of beta-nicotinamide adenine dinucleotide phosphate, reduced form (NADPH, 1 mM) and terminated by transferring an aliquot into solvent after different time points. Additionally, the NADPH-independent degradation was monitored in incubations without NADPH, terminated at the last time point. The [%] remaining test compound after NADPH independent incubation is reflected by the parameter c(control) (metabolic stability). The quenched incubations are pelleted by centrifugation (10000 g, 5 min).

An aliquot of the supernatant is assayed by LC-MS/MS for the amount of parent compound. The half-life (t1/2 INVITRO) is determined by the slope of the semilogarithmic plot of the concentration-time profile. The intrinsic clearance (CL_INTRINSIC) is calculated by considering the amount of protein in the incubation: CL_INTRINSIC [μl/min/mg protein]=(Ln 2/(half-life [min]*protein content [mg/ml]))*1000. For better across species comparison the predicted clearance is expressed as percent of the liver blood flow [% QH] in the individual species. In general, high stability (corresponding to low % QH) of the compounds across species is desired.

Mouse Liver Microsome (mLM) Assay

The metabolic degradation of the test compound is assayed at 37° C. with pooled liver microsomes from (male/female) mice (CD1). The final incubation volume of 100 μl per time point contains TRIS buffer pH 7.6 at RT (0.1 M), magnesium chloride (5 mM), microsomal protein (0.5 mg/ml) and the test compound at a final concentration of 1 μM. Following a short preincubation period at 37° C., the reactions were initiated by addition of beta-nicotinamide adenine dinucleotide phosphate, reduced form (NADPH, 1 mM) and terminated by transferring an aliquot into solvent after different time points. Additionally, the NADPH-independent degradation was monitored in incubations without NADPH, terminated at the last time point. The [%] remaining test compound after NADPH independent incubation is reflected by the parameter c(control) (metabolic stability). The quenched incubations are pelleted by centrifugation (10000 g, 5 min).

An aliquot of the supernatant is assayed by LC-MS/MS for the amount of parent compound. The half-life (t1/2 INVITRO) is determined by the slope of the semilogarithmic plot of the concentration-time profile. The intrinsic clearance (CL_INTRINSIC) is calculated by considering the amount of protein in the incubation: CL_INTRINSIC [μl/min/mg protein]=(Ln 2/(half-life [min]*protein content [mg/ml]))*1000. For better across species comparison the predicted clearance is expressed as percent of the liver blood flow [% QH] in the individual species. In general, high stability (corresponding to low % QH) of the compounds across species is desired.

Dog Hepatocyte (dHep) Assay

The metabolic degradation of the test compound is assayed in a suspension of dog hepatocyte cells.

Incubation: Cryopreserved dog hepatocyte cells are incubated in an appropriate buffer system (KHB buffer or similar buffer or standard cell culture medium) containing 50% species serum. Following an acclimation period (15-30 min) in an incubator (37° C., 5-10% CO2, 85-95% humidity) the test compound is added to the hepatocyte suspension (pH 7.4, typical cell density of about 1 million cells/mL; final concentration of test compound is 1 μM, final DMSO concentration <0.05% v/v). The cells are incubated for up to 6 hours and samples are taken at 6 different time points. Samples are then quenched with acetonitrile and pelleted by centrifugation. The remaining amount of parent compound in the supernatants is then analysed by HPLC-MS/MS.

Calculation: The elimination rate constant (ke) is calculated using the slope of the linear regression from natural log [substrate % remaining or substrate concentration] vs. time [h].

ke=(−1)*Slope
ke=elimination rate constant [1/h]
Half-life is calculated from the elimination rate constant
t½=In(2)/ke
t½=half-life [h]

Calculation of intrinsic in_vitro hepatic clearance:

CL_int_in vitro=ke*1000/(CD*60)
CL_int_in vitro=intrinsic hepatic clearance, in vitro [μl/min/Mio cells]
CD=Cell density [Mio cells/mL]
Note: the above equation is only true when [Substrate]<<Km.

The calculated in vitro hepatic intrinsic clearance can be scaled up to the intrinsic in vivo hepatic Clearance and used to predict hepatic in vivo blood clearance (CL_ws) by the use of a liver model (well stirred model).

CL_int_invivo=(CL_int_in vitro*H*L)/1000
CL_int_invivo=intrinsic hepatic clearance, in vivo [mL/min/kg]
H=hepatocellularity [Mio cells/g liver]
L=liver factor [g/kg body weight]

CL_ws=CL_int_invivo*Q/(CL_int_invivo+Q)
CL=Estimate of hepatic clearance, in vivo [mL/min/kg]
Q=hepatic blood flow [mL/min/kg]
ws=well stirred

QH%=CL_ws*100/Q
QH %=Clearance expressed as a percent of hepatic blood flow
CONC: cell concentration at incubation time (10\hat{ }6/ml)
T_LAST: terminal time point used (h)

Mouse Hepatocyte (mHep) Assay

The metabolic degradation of the test compound is assayed in a suspension of mouse hepatocyte cells.

Incubation: Cryopreserved mouse hepatocyte cells are incubated in an appropriate buffer system (KHB buffer or similar buffer or standard cell culture medium) containing 50% species serum. Following an acclimation period (15-30 min) in an incubator (37° C., 5-10% CO2, 85-95% humidity) the test compound is added to the hepatocyte suspension (pH 7.4, typical cell density of about 1 million cells/mL; final concentration of test compound is 1 μM, final DMSO concentration <0.05% v/v). The cells are incubated for up to 6 hours and samples are taken at 6 different time points. Samples are then quenched with acetonitrile and pelleted by centrifugation. The remaining amount of parent compound in the supernatants is then analysed by HPLC-MS/MS. Calculation: The elimination rate constant (ke) is calculated using the slope of the linear regression from natural log [substrate % remaining or substrate concentration] vs. time [h].

ke=(−1)*Slope
ke=elimination rate constant [1/h].
Half-life is calculated from the elimination rate constant
t½=In(2)/ke
t½=half-life [h]

Calculation of intrinsic in vitro hepatic clearance:

CL_int_in vitro=ke*1000/(CD*60)
CL_int_in vitro=intrinsic hepatic clearance, in vitro [μl/min/Mio cells]
CD=Cell density [Mio cells/mL]
Note: the above equation is only true when [Substrate]<<Km.

The calculated in vitro hepatic intrinsic clearance can be scaled up to the intrinsic in vivo hepatic Clearance and used to predict hepatic in vivo blood clearance (CL_ws) by the use of a liver model (well stirred model).

CL_int_invivo=(CL_int_in vitro*H*L)/1000
CL_int_invivo=intrinsic hepatic clearance, in vivo [mL/min/kg]
H=hepatocellularity [Mio cells/g liver]
L=liver factor [g/kg body weight]

CL_ws=CL_int_invivo*Q/(CL_int_invivo+Q)
CL=Estimate of hepatic clearance, in vivo [mL/min/kg]
Q=hepatic blood flow [mL/min/kg]
ws=well stirred

QH%=CL_ws*100/Q
QH %=Clearance expressed as a percent of hepatic blood flow
CONC: cell concentration at incubation time (10\hat{ }6/ml)
T_LAST: terminal time point used (h)
Hepatocellularity, mouse: 120×10e6 cells/g liver
Liver factor, mouse: 55 g/kg bodyweight
Blood flow, mouse: 90 ml/(min×kg).

Permeability Assay (MDCK-PGP)

The assay provides information on the potential of a compound to pass the blood brain barrier. Permeability measurements across polarized, confluent MDCK-MDR1 cell monolayers grown on permeable filter supports are used as the in vitro absorption model.

Apparent permeability coefficients (PE) of the compounds across the MDCK-MDR1 cell monolayers are measured (pH 7.4, 37° C.) in apical-to-basal (AB) and basal-to-apical (BA) transport direction. AB permeability (PEAB) represents drug absorption from the blood into the brain and BA permeability (PEBA) drug efflux from the brain back into the blood via both passive permeability as well as active transport mechanisms mediated by efflux and uptake transporters that are expressed on the MDCK-MDR1 cells, predominantly by the overexpressed human MDR1 P-gp. The compounds are assigned to permeability/absorption classes by comparison of the AB permeabilities with the AB permeabilities of reference compounds with known in vitro permeability and oral absorption in the human. Identical or similar permeabilities in both transport directions indicate passive permeation, vectorial permeability points to additional active transport mechanisms. Higher PEBA than PEAB indicates the involvement of active efflux mediated by MDR1 P-gp. Active transport is concentration-dependently saturable.

MDCK-MDR1 cells (1-2×10\hat{ }5 cells/1 cm\hat{ }2 area) are seeded on filter inserts (Costar transwell polycarbonate or PET filters, 0.4 μm pore size) and cultured (DMEM) for 7 days. Subsequently, the MDR1 expression is boosted by culturing the cells with 5 mM sodium butyrate in full medium for 2 days. Compounds are dissolved in appropriate solvent (like DMSO, 1-20 mM stock solutions). Stock solutions are diluted with HTP-4 buffer (128.13 mM NaCl, 5.36 mM KCl, 1 mM MgSO4, 1.8 mM CaCl2, 4.17 mM NaHCO3, 1.19 mM Na2HPO4×7H2O, 0.41 mM NaH2PO4×H2O, 15 mM HEPES, 20 mM glucose, 0.25% BSA, pH 7.4) to prepare the transport solutions (0.1-300 μM compound, final DMSO <=0.5%). The transport solution (TL) is applied to the apical or basolateral donor side for measuring A-B or B-A permeability (3 filter replicates), respectively. The receiver side contains the same buffer as the donor side. Samples are collected at the start and end of experiment from the donor and at various time intervals for up to 2 hours also from the receiver side for concentration measurement by HPLC-MS/MS or scintillation counting. Sampled receiver volumes are replaced with fresh receiver solution.

Results

TABLE 1
Interaction with dog STING as determined by DSF (dDSF)

| Example No. | Delta TM (° C.) |
|---|---|
| Example 1 | 34.0 |
| Example 2 | 33.3 |
| Example 3 | 26.1 |
| Example 4 | 29.0 |
| Example 5 | 33.5 |
| Example 6 | 34.0 |
| Example 7 | 15.5 |
| Example 8 | 24.8 |
| Example 9 | 33.8 |
| Example 10 | 35.8 |
| Example 11 | 28.3 |
| Example 13 | 28.3 |
| Example 14 | 23.8 |
| Example 15 | 30.0 |
| Example 18 | 31.5 |
| Example 19 | 33.5 |

TABLE 2
Cytokine secretion in dog whole blood (dWB) culture system
as determined by canine Interferon-beta (IFNb) ELISA

| Example No. | EC50 (nM) |
|---|---|
| Example 1 | 307.9 |
| Example 2 | 212.0 |
| Example 3 | 3063.5 |
| Example 4 | 703.9 |
| Example 5 | 529.0 |
| Example 6 | 416.8 |
| Example 9 | 651.6 |
| Example 10 | 184.9 |
| Example 11 | 619.7 |
| Example 13 | 281.6 |
| Example 14 | 2918.4 |
| Example 15 | 95.1 |
| Example 18 | 519.2 |
| Example 19 | 278.3 |

As demonstrated by the present examples, when measured with a binding assay, the compounds in accordance with the invention show an interaction with dog STING (dSTING) reflected by a shift in Tₘ of >15° C., more preferably >20° C., and even more preferably >25° C., as determined by DSF. Furthermore, the compounds in accordance with the present invention induce cytokine secretion in dog whole blood (dWB). In accordance with the invention, a combination of a high dDSF and low dWB is especially favorable.

1. A compound of formula (I) 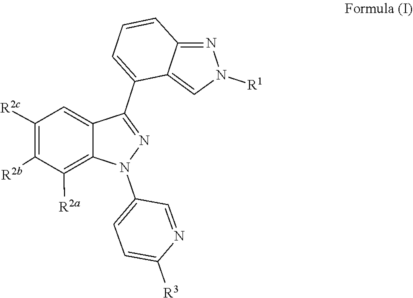
wherein
R¹ is a —C₁₋₆-alkyl or —C₃₋₆ cycloalkyl;
R^{2a} is selected from among the group consisting of —H or —C₁₋₆-alkyl;
R^{2b} is selected from among the group consisting of —H, —C₁₋₆-alkyl, —C₁₋₆-alkylene-OH, —C(O)OH, —C(O)O—C₁₋₆-alkyl and -pyrazolyl-C₁₋₆-alkyl;
R^{2c} is —H or —C₁₋₆-alkyl;
R³ is selected from among the group consisting of (* indicates the point of attachment): 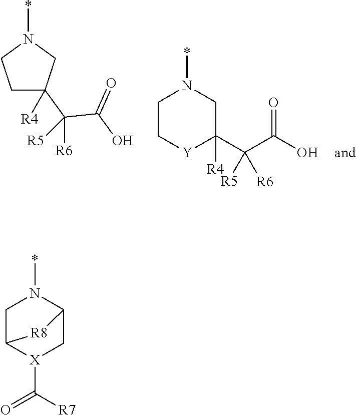
R⁴ is selected from H, —C₁₋₆-alkyl, and C₃₋₆ cycloalkyl;
R⁵ is selected from H, —C₁₋₆-alkyl, and C₃₋₆ cycloalkyl;
R⁶ is selected from H, —C₁₋₆-alkyl, and C₃₋₆ cycloalkyl;
R⁷ is selected from H, —C₁₋₆-alkyl, C₃₋₆ cycloalkyl and —OH;
R⁸ is (CH₂)ₙ, wherein n is an integer of 1-3, preferably 1 or 2;
X is CH or N; and Y is —O—, —S—, —S(O)—, —S(O)₂—;
or a pharmaceutically acceptable salt thereof.

2. A compound according to claim 1, wherein
R¹ is —C₁₋₆-alkyl, preferably methyl;
or a pharmaceutically acceptable salt thereof.

3. A compound according to claim 1, wherein
R², is —C₁₋₆-alkyl, preferably methyl;
R^{2b} is —H or —C₁₋₆-alkyl; and
R^{2c} is —H or —C₁₋₆-alkyl;
or a pharmaceutically acceptable salt thereof.

4. A compound according to claim 1, wherein
R², is methyl;
R^{2b} is —H; and
R^{2c} is —H;
or a pharmaceutically acceptable salt thereof.

5. A compound according to claim 1, wherein
R³ is 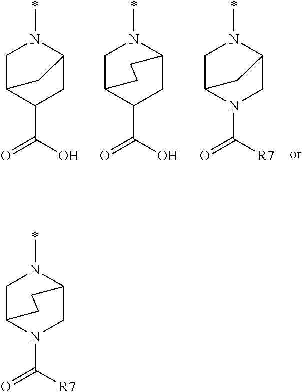
wherein R⁷ is —C₁₋₆-alkyl, preferably methyl;
or a pharmaceutically acceptable salt thereof.

6. A compound according to claim 1, wherein
R³ is 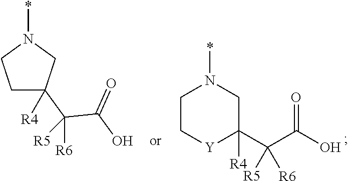
or a pharmaceutically acceptable salt thereof.

7. A compound according to claim 1, wherein
R³ is 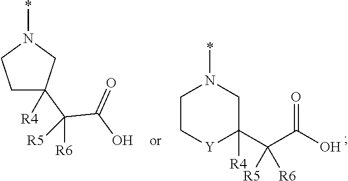
R⁴ is —H or methyl, preferably —H;
R⁵ and R⁶ are independently —H or methyl, preferably R⁵ and R⁶ are both —H; and
Y is O;
or a pharmaceutically acceptable salt thereof.

8. A compound according to claim 1, wherein
R¹ is methyl;
R², is methyl;
R^{2b} and R^{2c} are —H;
R³ is 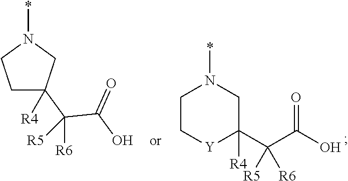
R⁴, R⁵ and R⁶ are —H; and
Y is O;
or a pharmaceutically acceptable salt thereof.

9. A compound which is selected from the following compounds: 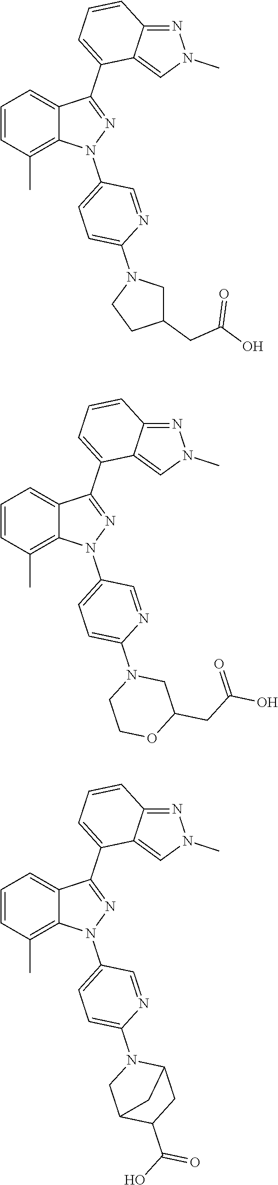 [SMILES: Cc1cccc2c(-c3cccc4nn(C)cc34)nn(-c3ccc(N4CCC(CC(=O)O)C4)nc3)c12 ; Cc1cccc2c(-c3cccc4nn(C)cc34)nn(-c3ccc(N4CCOC(CC(=O)O)C4)nc3)c12 ; Cc1cccc2c(-c3cccc4nn(C)cc34)nn(-c3ccc(N4CC5CC4CC5C(=O)O)nc3)c12] 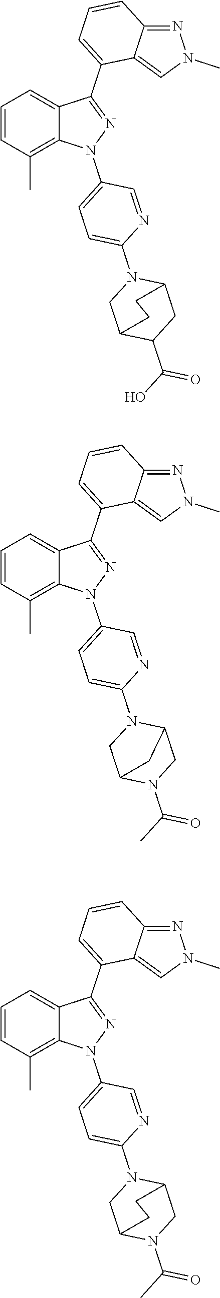 [SMILES: Cc1cccc2c(-c3cccc4nn(C)cc34)nn(-c3ccc(N4CC5CCC4CC5C(=O)O)nc3)c12 ; CC(=O)N1CC2CC1CN2c1ccc(-n2nc(-c3cccc4nn(C)cc34)c3cccc(C)c32)cn1 ; CC(=O)N1CC2CCC1CN2c1ccc(-n2nc(-c3cccc4nn(C)cc34)c3cccc(C)c32)cn1]
or a pharmaceutically acceptable salt thereof.

10. A compound which is selected from the following compounds: 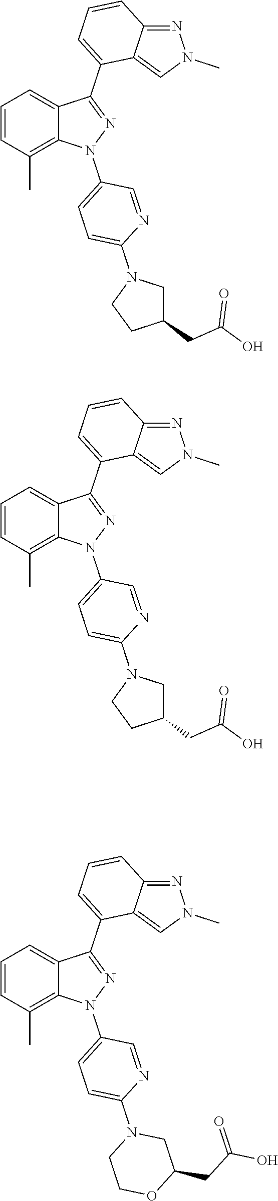 [SMILES: Cc1cccc2c(-c3cccc4nn(C)cc34)nn(-c3ccc(N4CC[C@H](CC(=O)O)C4)nc3)c12 ; Cc1cccc2c(-c3cccc4nn(C)cc34)nn(-c3ccc(N4CC[C@@H](CC(=O)O)C4)nc3)c12 ; Cc1cccc2c(-c3cccc4nn(C)cc34)nn(-c3ccc(N4CCO[C@H](CC(=O)O)C4)nc3)c12] 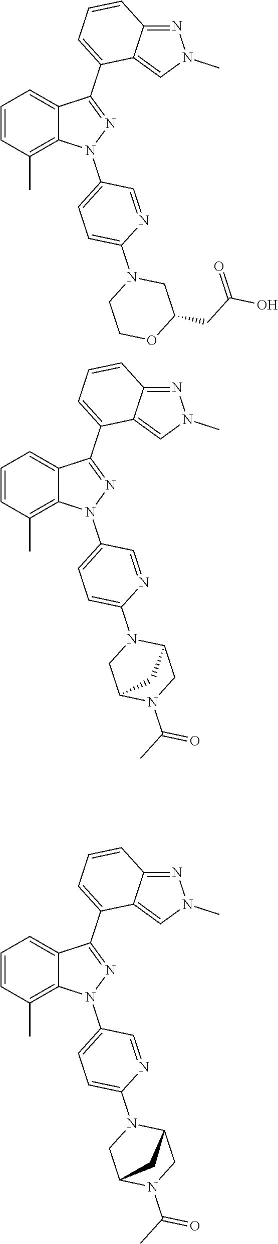 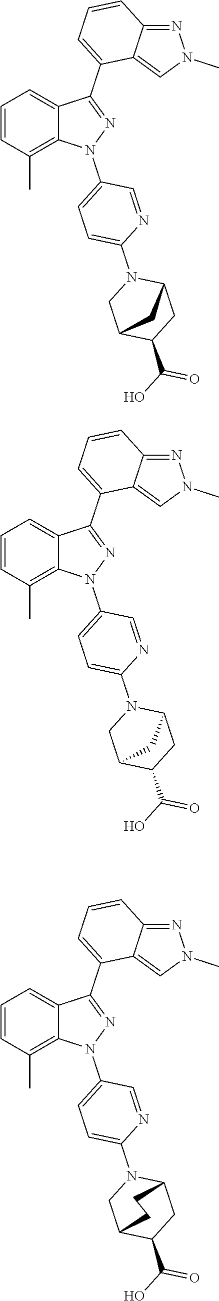 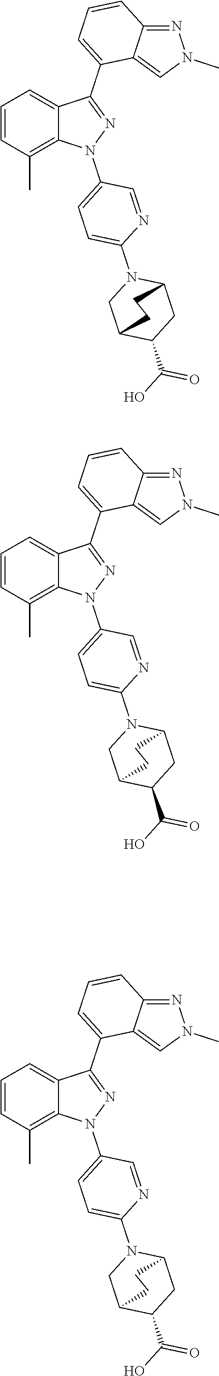 [SMILES: Cc1cccc2c(-c3cccc4nn(C)cc34)nn(-c3ccc(N4C[C@@H]5CC[C@H]4C[C@@H]5C(=O)O)nc3)c12 ; Cc1cccc2c(-c3cccc4nn(C)cc34)nn(-c3ccc(N4C[C@H]5CC[C@@H]4C[C@H]5C(=O)O)nc3)c12 ; Cc1cccc2c(-c3cccc4nn(C)cc34)nn(-c3ccc(N4C[C@H]5CC[C@@H]4C[C@@H]5C(=O)O)nc3)c12] 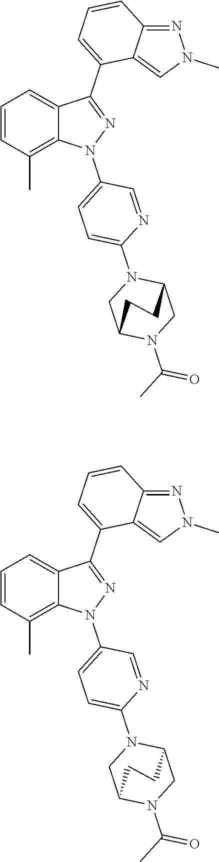 [SMILES: CC(=O)N1C[C@@H]2CC[C@H]1CN2c1ccc(-n2nc(-c3cccc4nn(C)cc34)c3cccc(C)c32)cn1 ; CC(=O)N1C[C@H]2CC[C@@H]1CN2c1ccc(-n2nc(-c3cccc4nn(C)cc34)c3cccc(C)c32)cn1]
or a pharmaceutically acceptable salt thereof.

11. A pharmaceutical composition comprising at least one compound of formula (I) according to claim 1, or a pharmaceutically acceptable salt thereof, and a pharmaceutically acceptable carrier.

12. A method for the treatment of a disease and/or condition in a mammal wherein the modulation of STING is of therapeutic benefit which comprises administering to said mammal an effective amount of a compound of formula (I) according to claim 1, or a pharmaceutically acceptable salt thereof.

13. A method according to claim 12, wherein the disease and/or condition is selected from the group consisting of inflammation, allergic or autoimmune diseases, infectious diseases and cancer.

14. A method according to claim 13, wherein the disease and/or condition is feline or canine cancer.

15. A method according to claim 14, wherein the disease and/or condition is canine cancer selected from osteosarcoma (OSA), oral melanoma, B-cell lymphoma, urothelial carcinoma (UC), hemangiosarcoma, mast cell tumor, soft tissue sarcoma, squamous cell carcinoma, T-cell lymphoma, mammary gland adenocarcinoma and anal sac carcinoma

16. A method according to claim 14, wherein the disease and/or condition is feline cancer is selected from B-cell and/or T-cell lymphoma, squamous cell carcinoma, mammary gland adenocarcinoma, mast cell tumors and injection site sarcoma.

17. A method according to claim 15, wherein the method is used in combination with radiotherapy.

18. A method according to claim 16, wherein the method is used in combination with radiotherapy.


SEQUENCE LISTING (1 sequence)

SEQ ID NO: 1 - protein, 244 aa, Canis sp.
MHHHHHHHHENLYFQSGEKRNFNVAHGLAWSYFIGYLRLILPGLPARIQALHNNMLQGIGSHRLHILFPLDCGVPDDLSVVDPNIRFLYELPQQSANRAGIKRRVYTNSVYELLEKGQPAGICVLEYATPLQTLFAMSQDGRAGFSREDRLEQAKLFCRTLEDILADAPELQNNCRLIVYQEPAEGSSFSLSQEILRHLRQEEREVTMGSMDTSIVPTSSTLSQEPNLFISGLEQPLPLRTDIF